SEQUENCE LISTING (22 sequences)

SEQ ID NO: 1 - protein, 5 aa, Artificial Sequence
  Description of Artificial Sequence Synthetic peptide
GGGGS

SEQ ID NO: 2 - protein, 6 aa, Artificial Sequence
  Description of Artificial Sequence Synthetic peptide
GGGGGG

SEQ ID NO: 3 - protein, 8 aa, Artificial Sequence
  Description of Artificial Sequence Synthetic peptide
GGGGGGGG

SEQ ID NO: 4 - protein, 18 aa, Artificial Sequence
  Description of Artificial Sequence Synthetic peptide
KESGSVSSEQLAQFRSLD

SEQ ID NO: 5 - protein, 14 aa, Artificial Sequence
  Description of Artificial Sequence Synthetic peptide
EGKSSGSGSESKST

SEQ ID NO: 6 - protein, 12 aa, Artificial Sequence
  Description of Artificial Sequence Synthetic peptide
GSAGSAAGSGEF

SEQ ID NO: 7 - protein, 5 aa, Artificial Sequence
  Description of Artificial Sequence Synthetic peptide
DDDDK

SEQ ID NO: 8 - protein, 25 aa, Artificial Sequence
  Description of Artificial Sequence Synthetic peptide
  MISC_FEATURE (1)..(25): This sequence may encompass 2-5 “Glu Ala Ala Ala Lys” repeating units
EAAAKEAAAKEAAAKEAAAKEAAAK

SEQ ID NO: 9 - protein, 27 aa, Artificial Sequence
  Description of Artificial Sequence Synthetic peptide
  MISC_FEATURE (2)..(26): This region may encompass 2-5 “Glu Ala Ala Ala Lys” repeating units
AEAAAKEAAAKEAAAKEAAAKEAAAKA

SEQ ID NO: 10 - protein, 10 aa, Artificial Sequence
  Description of Artificial Sequence Synthetic peptide
  MOD_RES (1)..(1): Any amino acid
  MISC_FEATURE (1)..(10): This sequence may encompass 2-5 “Xaa Pro” repeating units
  MOD_RES (3)..(3): Any amino acid
  MOD_RES (5)..(5): Any amino acid
  MOD_RES (7)..(7): Any amino acid
  MOD_RES (9)..(9): Any amino acid
XPXPXPXPXP

SEQ ID NO: 11 - protein, 5 aa, Artificial Sequence
  Description of Artificial Sequence Synthetic peptide
CPRSC

SEQ ID NO: 12 - protein, 9 aa, Unknown
  Description of Unknown Cytomegalovirus sequence
NLVPMVATV

SEQ ID NO: 13 - protein, 10 aa, Artificial Sequence
  Description of Artificial Sequence Synthetic peptide
  MOD_RES (1)..(1): N-formylmethionine
MNLVPMVATV

SEQ ID NO: 14 - protein, 9 aa, Artificial Sequence
  Description of Artificial Sequence Synthetic peptide
  MOD_RES (3)..(7): Any amino acid
NLXXXXXTV

SEQ ID NO: 15 - protein, 14 aa, Homo sapiens
CAADPSDSWGKLQF

SEQ ID NO: 16 - protein, 10 aa, Homo sapiens
CAFNDYKLSF

SEQ ID NO: 17 - protein, 14 aa, Homo sapiens
CAGPMKTSYDKVIF

SEQ ID NO: 18 - protein, 13 aa, Homo sapiens
CAVVDNYGQNFVF

SEQ ID NO: 19 - protein, 16 aa, Homo sapiens
CASSESRILTYNEQFF

SEQ ID NO: 20 - protein, 14 aa, Homo sapiens
CAWVPGTGGTEAFF

SEQ ID NO: 21 - protein, 12 aa, Homo sapiens
CASASANYGYTF

SEQ ID NO: 22 - protein, 11 aa, Homo sapiens
CASSSVGAWTF

(19) United States
(12) Patent Application Publication
(10) Pub. No.: US 2022/0090297 A1
(43) Pub. Date: Mar. 24, 2022

(54) PEPTIDE LIBRARIES AND METHODS OF USE THEREOF

(71) Applicant: REPERTOIRE IMMUNE MEDICINES, INC., Cambridge, MA (US)

(72) Inventors: Joanna Feltham Swain, Concord, MA (US); Ellen Lovisa Larsdotter Afzelius, Belmont, MA (US); William Michael Gordon, Way land, MA (US); Christian B. Matranga, Stow, MA (US); Assaf Rotem, Newton, MA (US); Gang Hu, Newton, MA (US)

(21) Appl. No.: 17/419,610

(22) PCT Filed: Jan. 3, 2020
(86) PCT No.: PCT/US2020/012236

§ 371 Date: Jun. 29, 2021

Related U.S. Application Data
(60) Provisional application No. 62/788,678, filed on Jan. 4, 2019.
(60) Provisional application No. 62/791,601, filed on Jan. 11, 2019.

(51) Int. Cl. C40B 50/14; C40B 40/10; C40B 30/04

(52) U.S. Cl. CPC C40B 50/14; C40B 30/04; C40B 40/10

(57) ABSTRACT

The disclosure relates to peptide libraries and uses thereof.

### CROSS REFERENCE

This application claims the benefit of U.S. Provisional Application No. 62/788,678, filed Jan. 4, 2019, and U.S. Provisional Application No. 62/791,601, filed Jan. 11, 2019, each of which is incorporated herein by reference in its entirety.

### BACKGROUND

There are a number of challenges related to the production and use of diverse peptide libraries.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The present disclosure provides compositions of peptide libraries and methods of use of these peptide libraries.

Disclosed herein, in some embodiments, is a peptide library comprising a plurality of peptides, wherein the plurality of peptides comprise more than 1,000, more than 2,000, more than 5,000, more than 10,000, more than 10⁶, more than 10⁷, more than 10⁸, more than 10⁹, or more than 10¹⁰ unique peptides.

Disclosed herein, in some embodiments, is a method of isolating lymphocyte-peptide pairs comprising: (a) contacting a plurality of lymphocytes with a peptide library, wherein the peptide library has a diversity of more than 1000; and (b) generating a plurality of compartments, wherein a compartment of the plurality comprises (i) a lymphocyte of the plurality of lymphocytes bound to a peptide of the peptide library, and (ii) a capture support.

Disclosed herein, in some embodiments, is a method of identifying a lymphocyte-peptide pair comprising: (a) contacting a plurality of lymphocytes with a library of peptides, wherein the library of peptides has a diversity of more than 1000; (b) compartmentalizing a lymphocyte of the plurality of lymphocytes bound to a peptide of the library of peptides in a single compartment, wherein the peptide comprises a unique peptide identifier; and (c) determining the unique peptide identifier for each peptide bound to the compartmentalized lymphocyte.

Disclosed herein, in some embodiments, is a method of using an unbiased peptide library, the method comprising contacting a sample with the unbiased peptide library comprising a plurality of peptides, wherein the plurality of peptides comprise more than 100, more than 1,000, more than 2,000, more than 5,000, more than 10,000, more than 10⁶, more than 10⁷, more than 10⁸, more than 10⁹, or more than 10¹⁰ unique peptides.

Disclosed herein, in some embodiments, is a composition comprising pMHC multimer attached to a unique identifier.

Disclosed herein, in some embodiments, is a compartment comprising: (a) a sequence encoding a sc-pMHC; and (b) a T cell.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides exemplary embodiments of peptide libraries of various diversities.

FIG. 2A demonstrates enzymatic cleavage to increase peptide diversity. Lane pairs 1-2, 3-4, 5-6, 7-8 and 9-10 represent cell-free protein synthesis (CFPS) reactions performed on a template without a cleavage moiety, a template with a cleavage moiety where no protease was added, a template with a cleavage moiety in which the protease was added after the reaction was completed, a template with a cleavage moiety in which the protease was present during the reaction and a reaction that lacked a template, respectively. Western blotting was performed to determine total protein yield.

FIG. 2B show samples from the CFPS reaction that contained multimer and monomer templates with or without the cleavage moiety. The samples were blotted and detected with an anti-FLAG HRP antibody.

FIG. 2C demonstrates that peptides generated by CFPS and subjected to proteolytic cleavage fold into a recognizable 3-dimensional structure. CFPS protein was tested for conformational recognition by an antibody. The figure indicates whether protease-cleaved peptide or the uncleaved (fMet-containing) peptide demonstrated correct folding by recognition with the conformational epitope antibody.

FIG. 2D provides binding of a single-chain peptide-MHC (sc-pMHC) multimer to antigen-specific T cells. Multimers were produced by CFPS and protease cleavage. T cells were incubated with multimers, then stained with a fluorescent detection antibody and analyzed by flow cytometry.

FIG. 3 is a blot showing peptides labeled with one or more peptide identifiers as demonstrated by an upward shift of the peptide band.

FIG. 4 shows flow cytometry results of the relative binding of naked CMV peptide, CMV peptide labeled with peptide identifier, or negative control to CMV or non-CMV specific T cells.

FIG. 5 shows qPCR results of the relative amount of peptide identifier detected after binding peptide to T cells. The quantities were normalized to a housekeeping gene.

FIG. 6 illustrates enrichment of folded peptides in a library of the disclosure.

FIG. 7 illustrates validation of library diversity based on sequencing of nucleic acid identifiers.

FIG. 8 illustrates identification of peptide (antigen)-receptor (TCR) pairs by analysis of sequencing data generated using compositions and methods of the disclosure.

FIG. 9 illustrates analysis of peptide (antigen)-receptor (TCR) pair data generated using compositions and methods of the disclosure. FIG. 9A illustrates clustering by TCR to show antigen specificity profiles. FIG. 9B illustrates tSNE clustering by antigen to show TCR binding convergence. FIG. 9C illustrates inter-subject convergence of diverse TCRs.

FIG. 10 illustrates binding of sc-pMHC to TCR and the effect of antigen mutations on binding.

FIG. 11 illustrates exemplary immunophenotyping based on gene expression of cells of interest identified using the methods described herein.

FIG. 12 illustrates specific and dose-dependent binding of sc-pMHC to antigen-specific T cells for an exemplary antigen identified using the methods described herein.

FIG. 13 illustrates proliferation and cytokine production of T cells in response to cognate sc-pMHC for an exemplary antigen identified using the methods described herein.

FIG. 14 shows exemplary library sizes. FIG. 14A shows exemplary virome library sizes.

FIG. 14B shows exemplary cancer library sizes.

FIG. 15 shows an exemplary overview of production of a peptide library of the present disclosure (for example, according to examples 21-25).

FIG. 16 provides a schematic of a nucleic acid construct that can be used in compositions and methods of the disclosure. The construct can encode a peptide of the disclosure (e.g., a sc-pMHC) and an identifier (e.g., a self-identifier that corresponds to all or a part of the coding sequence of the peptide). The locations of forward and reverse primers are indicated (e.g., primers that can be used in examples 21-23).

FIG. 17 demonstrates PCR amplification of full length antigen-encoding templates (FIG. 17A) and identifiers (FIG. 17B) onto hydrogels as outlined in examples 22 and 23.

FIG. 18 demonstrates the generation of folded and identifier-tagged sc-pMHC of the disclosure. FIG. 18A provides microscopy images that demonstrate a sc-pMHC of the disclosure was in vitro transcribed and translated as outlined in example 24. FIG. 18B provides ELISA results that demonstrate release of folded sc-pMHC multimers as outlined in example 25. FIG. 18C provides a Western Blot that demonstrates the sc-pMHC is tagged with an identifier as outlined in example 25.

FIG. 19 provides the results of a flow cytometry assay that demonstrates sc-pMHC produced by methods of the disclosure bind specifically to cognate T cells. as outlined in example 26.

FIG. 20 provides the results of a single cell sequencing assay that demonstrates sc-pMHC produced by methods of the disclosure bind specifically to cognate T cells. as outlined in example 26.

### DETAILED DESCRIPTION

### Definitions

As used herein, the term “identifier” refers to a readable representation of data that provides information, such as an identity, that corresponds with the identifier.

As used herein, the term “multimer” refers to a plurality of units. In some embodiments, the multimer comprises one or more different units. In some embodiments, the units in the multimer are the same. In some embodiments, the units in the multimer are different. In some embodiments, the multimer comprises a mixture of units that are the same and different.

As used herein, the term “peptide library” refers to a plurality of peptides. In some embodiments, the library comprises one or more peptides with unique sequences. In some embodiments, each peptide in the library has a different sequence. In some embodiments, the library comprises a mixture of peptides with the same and different sequences.

As used herein, the term “unbiased” refers to lacking one or more selective criteria.

As used herein, the term “capture support” refers to an interaction surface. In some embodiments, a capture support can be a solid surface. In some embodiments, a capture support can comprise a matrix. In some embodiments, a capture support can comprise a nanoparticle. In some embodiments, a capture support can comprise a bead. In some embodiments, a capture support can comprise a magnetic bead. In some embodiments, a capture support can comprise a hydrogel. In some embodiments, a capture support can be the inner surface of a water in oil emulsion droplet. In some embodiments, a capture support can comprise a nucleic acid molecule. In some embodiments, a capture support can comprise a protein. In some embodiments, a capture support can comprise an antibody or derivative thereof. In some embodiments, a capture support can comprise a gel. In some embodiments, a capture support can comprise a polymer. In some embodiments, a capture support can be charged. In some embodiments, a capture support can be fluorescent, e.g., labelled with a fluorescent dye or dyes.

### INTRODUCTION

This disclosure provides a peptide library. A peptide library can comprise a plurality of peptides with different amino acid sequences. Peptide libraries can be used in a range of screening assays to identify potential diagnostic or therapeutic targets or agents. Peptide libraries can be used, for example, to screen for disease-specific, organ-specific, or other compartment-specific peptides, to screen for peptides with therapeutic applications, to screen for peptides with diagnostic applications, to screen for tumor-targeting peptides, to screen for antibody epitopes or antigens, to screen for T cell epitopes or antigens, to screen for antimicrobial peptides, or any combination thereof. Diverse peptide libraries of appropriate quality, therefore, have many valuable uses.

A peptide library can be an antigen library. Non-limiting examples of applications of antigen libraries include use in assays, therapies, and diagnostics related to immune antigens. For example, antigen libraries can be used in screens to identify a protein epitope, such as an antibody or T cell epitope. Antibody and T cell epitopes can greatly impact the function of the adaptive immune system, as they are the specific sequences that antibodies, B cell receptors (BCRs), and T cell receptors (TCRs) recognize. Many effector mechanisms of the immune system can be triggered by antibody epitopes or T cell epitopes, thus antigen libraries have potential applications including, but not limited to, in infectious disease, cancer immunotherapy, and autoimmunity. Antibody and T cell epitopes can be adapted for a wide range of uses, including, for example, the production of antibodies or antigen-binding fragments for therapeutic or laboratory use, the production of vaccines using antibody or T cell epitopes, and engineering T cells of known antigen specificity, e.g., for cancer immunotherapy.

There are a number of challenges related to the production and use of diverse peptide libraries. The sheer number of possible peptide sequences can present challenges for library production. For a peptide of 9 residues in length (a 9-mer), there are 20⁹ (approximately 5.1×10¹¹) possible sequence combinations of the 20 amino acids most commonly found in proteins. Potential antigen peptide sources of interest (e.g., pathogens, cancer cells, tissues) can express hundreds or thousands of proteins, each protein comprising a plurality of potential peptide antigens.

A number of techniques have been developed to generate peptides, however many have limitations (e.g., lack sufficient coverage of peptide diversity, are skewed toward high affinity interactions, use conditions for peptide generation that lead to denatured or misfolded proteins, etc.). This disclosure provides peptide libraries, including, for example, high diversity peptide libraries with useful applications as described herein. Also provided are methods of peptide library production, including, for example, methods for producing unbiased peptide libraries.

### Compositions of a Peptide Library

This disclosure provides peptide libraries, including, for example, high diversity peptide libraries useful in a range of therapeutic, diagnostic, and research applications. The peptide libraries provided can be used, for example, to screen for disease-specific, organ-specific, or other compartment-specific peptides, to screen for peptides with therapeutic applications, to screen for peptides with diagnostic applications, to screen for tumor-targeting peptides, to screen for antibody epitopes or antigens, to screen for T cell epitopes or antigens, to screen for antimicrobial peptides, or any combination thereof.

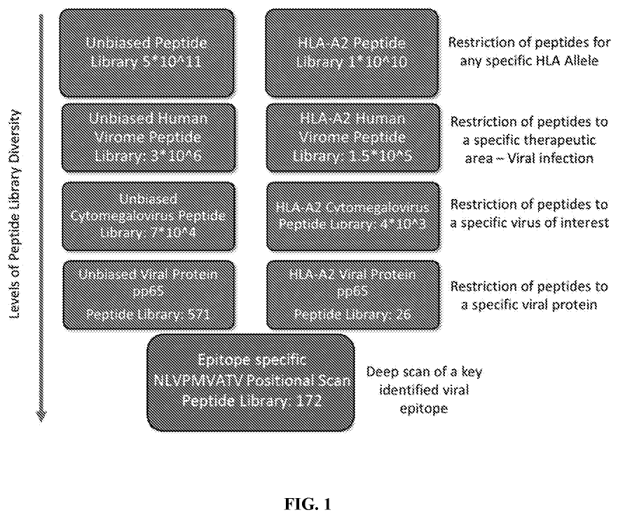

A peptide library of the disclosure can be unbiased or biased and can comprise any number of peptides. Exemplary embodiments of peptide libraries of various diversities are provided in FIG. 1 and described below.

### Unbiased Peptide Library

A peptide library disclosed herein can be an unbiased library, e.g., lacking one or more selection criteria. In some embodiments, a library of the disclosure comprises all possible combinations of amino acids for a peptide of a given size, the 20^{k} possible k-mer peptides comprising any of the standard 20 amino acids, for example, the 20⁹ possible 9-mer peptides comprising any of the standard 20 amino acids.

In some embodiments, the library comprises peptides having a range of lengths, e.g., from about 2 amino acids (2-mers) to about 20 amino acids (20-mers), or any suitable range. In some embodiments, the library comprises peptides having substantially the same length, e.g., 2-mers, 3-mers, 4-mers, 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, or longer.

In some embodiments, a library of the disclosure comprises constrained residues at certain positions, for example, constrained residues at positions 2 and 9 for docking to HLA-A2. In some embodiments, a library of the disclosure comprises all possible combinations of amino acids at the non-constrained residues, for example, the 20⁶ possible 6-mer sequences at positions 3, 4, 5, 6, 7, and 8 of a 9-mer sequence, with constrained residues at positions 1, 2, and 9. In some embodiments, a library of the disclosure comprises all possible combinations of amino acids in a peptide of a given size apart from constrained residues, for example, the 20⁷ possible 9-mer sequences when positions 2 and 9 are constrained, and positions 1, 3, 4, 5, 6, 7, and 8 are varied.

Constrained residues can be constrained to a single residue or to any subset of residues (e.g., constrained to valine, leucine or isoleucine). In some embodiments, constrained residues can be any one of a subclass of amino acids, for example, any hydrophobic amino acid, any hydrophilic amino acid, any charged amino acid, any basic amino acid, any acidic amino acid, any cyclic amino acid, any aromatic amino acid, any aliphatic amino acid, any polar amino acid, any non-polar amino acid, or any combination thereof varied residues can be selected from all possible residues or can be selected from any subset of residues. For example, any hydrophobic amino acid, any hydrophilic amino acid, any charged amino acid, any basic amino acid, any acidic amino acid, any cyclic amino acid, any aromatic amino acid, any aliphatic amino acid, any polar amino acid, any non-polar amino acid, or any combination thereof.

In some embodiments, a library of the disclosure comprises all k-mer peptides that can be made by any in silico production methods. In some embodiments, the library may comprise k-mer peptides from any translation product, e.g., epitope, antigen, protein, or proteome. In some embodiments, the library comprises k-mer peptides derived in silico from one or more genomes, exomes, transcriptomes, proteomes, ORFeomes, or any combinations thereof.

In some embodiments, the library comprises all k-mer peptides produced by transcription and translation of any polynucleotide sequence of interest, for example, in silico production of the transcription and translation products of both the forward and reverse strands of a genome or metagenome in all six reading frames. In some embodiments, a library of the disclosure comprises all k-mer peptides that can be derived from in silico transcription and translation of a mammalian genome, for example, a mouse genome, a human genome, a patient genome, an autoimmune patient genome, or a cancer genome. In some embodiments, a library of the disclosure comprises all k-mer peptides that can be derived from in silico transcription and translation of a microorganism genome, for example, a bacterial genome, a viral genome, a protozoan genome, a protist genome, a yeast genome, an archaeal genome, or a bacteriophage genome. In some embodiments, a library of the disclosure comprises all k-mer peptides that can be derived from in silico transcription and translation of a pathogen genome, for example, a bacterial pathogen genome, a viral pathogen genome, a fungal pathogen genome, an opportunistic pathogen genome, a conditional pathogen genome, or a eukaryotic parasite genome. In some embodiments, a library of the disclosure can be derived from a plant genome or a fungal genome. In some embodiments, a library of the disclosure comprises k-mer peptides derived from in silico transcription and translation of a genome, wherein the genome is modified during in silico transcription and translation, for example, in silico mutated to produce k-mer peptides comprising mutations (e.g., substitutions, insertions, deletions).

In some embodiments, a library of the disclosure comprises all k-mer peptides that can be derived from in silico translation of an exome of interest, for example, a mammalian exome, a human exome, a mouse exome, a patient exome, an autoimmune patient exome, a cancer exome, a viral exome, a protozoan exome, a protist exome, a yeast exome, a pathogen exome, a eukaryotic parasite exome, a plant exome, or a fungal exome. In some embodiments, a library of the disclosure comprises k-mer peptides derived from in silico translation of an exome, wherein the exome is modified during in silico translation, for example, in silico mutated to produce k-mer peptides comprising mutations (e.g., substitutions, insertions, deletions).

In some embodiments, a library of the disclosure comprises all k-mer peptides that can be derived from in silico translation of a transcriptome of interest, for example, a mammalian transcriptome, a human transcriptome, a mouse transcriptome, a patient transcriptome, an autoimmune patient transcriptome, a cancer transcriptome, a microorganism transcriptome, a bacterial transcriptome, a viral transcriptome, a protozoan transcriptome, a protist transcriptome, a yeast transcriptome, an archaeal transcriptome, a bacteriophage transcriptome, a pathogen transcriptome, a eukaryotic parasite transcriptome, a plant transcriptome, a fungal transcriptome, a transcriptome derived from RNA sequencing, a microbiome transcriptome, or a transcriptome derived from metagenomic RNA-sequencing. In some embodiments, a library of the disclosure comprises k-mer peptides derived from in silico translation of a transcriptome, wherein the transcriptome is modified during in silico translation, for example, in silico mutated to produce k-mer peptides comprising mutations (e.g., substitutions, insertions, deletions).

In some embodiments, a library of the disclosure comprises all k-mer peptides that can be derived from a proteome of interest, for example, a mammalian proteome, a human proteome, a mouse proteome, a patient proteome, an autoimmune patient proteome, a cancer proteome, a microorganism proteome, a bacterial proteome, a viral proteome, a protozoan proteome, a protist proteome, a yeast proteome, an archaeal proteome, a bacteriophage proteome, a pathogen proteome, a eukaryotic parasite proteome, a plant proteome or a fungal proteome. In some embodiments, a library of the disclosure comprises k-mer peptides derived from a proteome wherein the k-mer peptides are modified from the proteome sequence, for example, k-mer peptides comprising mutations (e.g., substitutions, insertions, deletions).

In some embodiments, a library of the disclosure comprises all k-mer peptides that can be derived from in silico translation of an ORFeome of interest, for example, a mammalian ORFeome, a human ORFeome, a mouse ORFeome, a patient ORFeome, an autoimmune patient ORFeome, a cancer ORFeome, a microorganism ORFeome, a bacterial ORFeome, a viral ORFeome, a protozoan ORFeome, a protist ORFeome, a yeast ORFeome, an archaeal ORFeome, a bacteriophage ORFeome, a pathogen ORFeome, a eukaryotic parasite ORFeome, a plant ORFeome or a fungal ORFeome, an ORFeome derived from next-gen sequencing, a microbiome ORFeome, or an ORFeome derived from metagenomic sequencing. In some embodiments, a library of the disclosure comprises k-mer peptides derived from in silico translation of an ORFeome, wherein the ORFeome is modified during in silico translation, for example, in silico mutated to produce k-mer peptides comprising mutations (e.g., substitutions, insertions, deletions).

In some embodiments, a library of the disclosure comprises all k-mer peptides that can be derived from in silico transcription and translation or translation of a group of genomes, proteomes, transcriptomes, ORFeomes, or any combination thereof. In some embodiments, a library of the disclosure comprises all k-mer peptides that can be derived from in silico transcription and translation or translation of polynucleotide sequences from a group of samples, for example, clinical samples from a patient population, or a group of pathogen genomes. In some embodiments, a library of the disclosure comprises all k-mer peptides that can be derived from in silico transcription and translation of a group of viral genomes, for example, the human virome. In some embodiments, a library of the disclosure comprises all k-mer peptides that can be derived from in silico transcription and translation of a group of genomes, proteomes, transcriptomes, ORFeomes, or any combination thereof, wherein the source sequences are modified during in silico translation, for example, in silico mutated to produce k-mer peptides comprising mutations (e.g., substitutions, insertions, deletions).

In some embodiments, a library of the disclosure comprises all k-mer peptides that can be derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them), for example, differing in nucleotide sequence, amino acid sequence, nucleotide abundance, or protein abundance. In some embodiments, differential sequences of a genome, proteome, transcriptome, or ORFeome are generated by comparing tissues of interest. In some embodiments, differential sequences of a genome, proteome, transcriptome, or ORFeome are generated by comparing sequences from cells of interest (e.g., a healthy cell versus a cancer cell).

In some embodiments, differential sequences of a genome, proteome, transcriptome, or ORFeome are generated by comparing sequences of organisms of interest. In some embodiments, differential sequences of a genome, proteome, transcriptome, or ORFeome can be generated by comparing subjects of interest (e.g., diseased versus healthy subjects). In some embodiments, the differential sequences are different by at least 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, 5%, or 1%. In some embodiments, the differential sequences are different by from 1% to 100%, 5% to 100%, 10% to 100%, 15% to 100%, 20% to 100%, 25% to 100%, 30% to 100%, 40% to 100%, 50% to 100%, 60% to 100%, 70% to 100%, 80% to 100%, 90% to 100%, 95% to 100%, 30% to 90%, 40% to 90%, 50% to 90%, 60% to 90%, 70% to 90%, 80% to 90%, or 60% to 80%. In some embodiments, the differential sequences have at least 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 6%, 60%, 55%, 50%, 45%, 40%, 35%, 30%, 25%, 20%, 15%, 10%, 5%, or 1% difference between the compared sequences. In some embodiments, the differential sequences have from 1% to 100%, 5% to 100%, 10% to 100%, 15% to 100%, 20% to 100%, 25% to 100%, 30% to 100%, 40% to 100%, 50% to 100%, 60% to 100%, 70% to 100%, 80% to 100%, 90% to 100%, 95% to 100%, 30% to 90%, 40% to 90%, 50% to 90%, 60% to 90%, 70% to 90%, 80% to 90%, or 60% to 80% difference between the compared sequences.

In some embodiments, a library of the disclosure comprises all k-mer peptides that can be derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences (e.g., that share a degree of homology), for example, homologous nucleotide sequences, homologous amino acid sequences, homologous nucleotide abundance, or homologous protein abundance. In some embodiments, homologous sequences of genomes, proteomes, transcriptomes, or ORFeomes are generated by comparing tissues of interest. In some embodiments, homologous sequences of genomes, proteomes, transcriptomes, or ORFeomes are generated by comparing sequences from cells of interest (e.g., a healthy cell versus a involved in autoimmunity cell (e.g., a cell that induces autoimmunity or a cell that is targeted during autoimmunity). In some embodiments, homologous sequences of genomes, proteomes, transcriptomes, or ORFeomes are generated by comparing sequences of organisms of interest. In some embodiments, homologous sequences of genomes, proteomes, transcriptomes, or ORFeomes are generated by comparing subjects of interest (e.g., diseased versus healthy subjects). In some embodiments, the homologous sequences have a homology of at least 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, 5%, or 1%. In some embodiments, the homologous sequences have homology of from 1% to 100%, 5% to 100%, 10% to 100%, 15% to 100%, 20% to 100%, 25% to 100%, 30% to 100%, 40% to 100%, 50% to 100%, 60% to 100%, 70% to 100%, 80% to 100%, 90% to 100%, 95% to 100%, 30% to 90%, 40% to 90%, 50% to 90%, 60% to 90%, 70% to 90%, 80% to 90%, or 60% to 80%.

In some embodiments, a library of the disclosure comprises all k-mer peptides encoding a certain degree of homology to a sequence of interest (e.g., a differential sequence or homologous sequence identified as described above). In some embodiments, a library of the disclosure comprises the closest homologs between two or more sequences of interest.

In some embodiments, a library of the disclosure comprises all k-mer peptides that can be derived from a polypeptide sequence of interest, for example, all possible 9-mer peptides covering the complete protein sequence of a viral protein. In some embodiments, a library of the disclosure comprises k-mer peptides that can be generated from a polypeptide sequence of interest, wherein the polypeptide sequence of interest is modified, e.g., in silico mutated to produce k-mer peptides comprising mutations (e.g., substitutions, insertions, deletions).

In some embodiments, a library of the disclosure comprises all k-mer peptides that can be derived from mutations in a sequence of interest, for example, all 9-mer peptides that can be generated from single nucleotide mutations in a polynucleotide sequence encoding an antigen or epitope. For example, a library of the disclosure comprises all 9-mer peptides that can be generated from two, three, four, five, six, seven, eight, or nine nucleotide mutations in a polynucleotide sequence encoding an antigen or epitope. In some embodiments, a library of the disclosure comprises all k-mer peptides that can be derived from alanine substitutions, for example, alanine substitutions at any position in any of the sequences described herein (e.g., a protein, a group of proteins, a proteome, an in silico transcripted and translated genome). In some embodiments, a library of the disclosure comprises a positional scanning library, wherein selected amino acid residues are sequentially substituted with all other natural amino acids. In some embodiments, a library of the disclosure comprises a combinatorial positional scanning library, wherein selected amino acid residues are sequentially substituted with all other natural amino acids, two or more positions at a time. In some embodiments, a library of the disclosure comprises an overlapping peptide library, comprising overlapping peptides from a template sequence (e.g., in silico translated genome), wherein overlapping peptides of a set length are offset by a defined number of residues. In some embodiments, a library of the disclosure comprises a T cell truncated peptide library, wherein each replicate of the library comprises equimolar mixtures of peptides with truncations at one terminus (e.g., 8-mers, 9-mers, 10-mers and 11-mers that can be derived from C-terminal truncations of a nominal 11-mer). In some embodiments, a library of the disclosure comprises a customized set of peptides, wherein the customized set of peptides are provided in a list.

In some embodiments, a genome, exome, transcriptome, proteome, or ORFeome of the disclosure is a viral genome, exome, transcriptome, proteome, or ORFeome. Non-limiting examples of viruses include Adenovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Cytomegalovirus (CMV), Dengue virus, Dhori virus, Dugbe virus, Duvenhage virus, Eastern equine encephalitis virus, Ebolavirus, Echovirus, Encephalomyocarditis virus, Epstein-Barr virus (EBV), European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human endogenous retrovirus (HERV), Human enterovirus, Human herpesvirus (e.g., HHV-1, HHV-2, HHV-6A, HHV-6B, HHV-7, HHV-8, Human immunodeficiency virus (e.g., HIV-1, HIV-2), Human papillomavirus (e.g., HPV-1, HPV-2, HPV-16, HPV-18, Human parainfluenza, Human parvovirus B19, Human respiratory syncytial virus (RSV), Human rhinovirus, Human SARS coronavirus, Human spumaretrovirus, Human T-lymphotropic virus (HTLV, e.g., HTLV-1, HTLV-2, HTLV-3), Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norovirus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus (e.g., rotavirus A, rotavirus B, rotavirus C, rotavirus X), Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, a genome, exome, transcriptome, proteome, or ORFeome of the disclosure is a cancer genome, exome, transcriptome, proteome, or ORFeome. In some embodiments, a library of the disclosure comprises known cancer neoepitopes. In some embodiments, a library of the disclosure comprises all k-mer peptides that can be derived from known cancer antigenic proteins. In some embodiments, a library of the disclosure comprises all k-mer peptides that can be derived from genes involved in epithelial-mesenchymal transition. In some embodiments, a library of the disclosure comprises all k-mer peptides that can be derived from cancer implicated genes. In some embodiments, a library of the disclosure comprises all k-mer peptides that can be derived from mutational cancer driver genes. In some embodiments, a library of the disclosure comprises all k-mer peptides that can be derived from proto-oncogenes, oncogenes, or tumor suppressor genes. In some embodiments, a library of the disclosure comprises all k-mer peptides that can be derived from proto-oncogenes, oncogenes, or tumor suppressor genes, wherein the k-mers comprise mutations as described herein (e.g., amino acid substitutions, alanine substitutions, positional scanning, combinatorial positional scanning etc.).

Non-limiting examples of cancers include Acute Lymphoblastic Leukemia (ALL), Acute Myeloid Leukemia (AML), Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, Bone Cancer, Brain Tumor, Breast Cancer, Bronchial Tumors, Burkitt Lymphoma, Carcinoid Tumor, Carcinoma of Unknown Primary, Cardiac Tumor, Central Nervous System cancer, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumor, Endometrial Cancer, Epithelial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumors, Gestational Trophoblastic Disease, Hairy Cell Leukemia, Head and Neck Cancer, Hepatocellular Cancer, Histiocytosis, Hodgkin Lymphoma, Hypopharyngeal Cancer, Intraocular Melanoma, Islet Cell Tumors, Kaposi Sarcoma, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Merkel Cell Carcinoma, Mesothelioma, Metastatic Cancer, Metastatic Squamous Neck Cancer with Occult Primary, Midline Tract Carcinoma, Mouth Cancer, Multiple Endocrine Neoplasia Syndrome, Multiple Myeloma, Mycosis Fungoides, Myelodysplastic Syndromes, Myelodysplastic/Myeloproliferative Neoplasms, Nasal Cavity Cancer, Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Osteosarcoma, Ovarian Cancer, Pancreatic Cancer, Pancreatic Neuroendocrine Tumors, Papillomatosis, Paraganglioma, Paranasal Sinus Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Stomach Cancer, T-Cell Lymphoma, Testicular Cancer, Throat Cancer, Thymoma and Thymic Carcinoma, Thyroid Cancer, Transitional Cell Cancer, Ureter and Renal Pelvis Cancer, Urethral Cancer, Uterine Cancer, Uterine Sarcoma, Vaginal Cancer, Vascular Tumors, Vulvar Cancer, and Wilms Tumor.

In some embodiments, a genome, exome, transcriptome, proteome, or ORFeome of the disclosure is an inflammatory or autoimmunogenic genome, exome, transcriptome, proteome, or ORFeome. In some embodiments, a library of the disclosure comprises known inflammatory or autoimmunogenic neoepitopes or self-epitopes. In some embodiments, a library of the disclosure comprises all k-mer peptides that can be derived from known inflammatory or autoimmunogenic antigenic proteins. In some embodiments, a library of the disclosure comprises all k-mer peptides that can be derived from inflammatory or autoimmune-implicated genes. In some embodiments, a library of the disclosure comprises all k-mer peptides that can be derived from mutation of inflammatory or autoimmune-related driver genes.

Non-limiting examples of inflammatory or autoimmune diseases or conditions include Acute Disseminated Encephalomyelitis (ADEM); Acute necrotizing hemorrhagic leukoencephalitis; Addison's disease; Adjuvant-induced arthritis; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune gastric atrophy; Autoimmune hemolytic anemia; Autoimmune hepatitis; Autoimmune hyperlipidemia; Autoimmune immunodeficiency; Autoimmune inner ear disease (AIED); Autoimmune myocarditis; Autoimmune oophoritis; Autoimmune pancreatitis; Autoimmune retinopathy; Autoimmune thrombocytopenic purpura (ATP); Autoimmune thyroid disease; Autoimmune urticarial; Axonal & neuronal neuropathies; Balo disease; Behcet's disease; Bullous pemphigoid; Cardiomyopathy; Castleman disease; Celiac disease; Chagas disease; Chronic inflammatory demyelinating polyneuropathy (CIDP); Chronic recurrent multifocal ostomyelitis (CRMO); Churg-Strauss syndrome; Cicatricial pemphigoid/benign mucosal pemphigoid; Crohn's disease; Cogans syndrome; Collagen-induced arthritis; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic esophagitis; Eosinophilic fasciitis; Erythema nodosum Experimental allergic encephalomyelitis; Experimental autoimmune encephalomyelitis; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis (GPA) (formerly called Wegener's Granulomatosis); Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropathy; IgG4-related sclerosing disease; Immunoregulatory lipoproteins; Inclusion body myositis; Interstitial cystitis; Inflammatory bowel disease; Juvenile arthritis; Juvenile oligoarthritis; Juvenile diabetes (Type 1 diabetes); Juvenile myositis; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Non-obese diabetes; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Pemphigus vulgaris; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatic; Polymyositis; Postmyocardial infarction syndrome; Postpericardiotomy syndrome; Progesterone dermatitis; Primary biliary cirrhosis; Primary sclerosing cholangitis; Psoriasis; Plaque Psoriasis; Psoriatic arthritis; Idiopathic pulmonary fibrosis; Pyoderma gangrenosum; Pure red cell aplasia; Raynauds phenomenon; Reactive Arthritis; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sclerosing cholangitis; Sclerosing sialadenitis; Sjogren's syndrome; Sperm & testicular autoimmunity; Stiff person syndrome; Subacute bacterial endocarditis (SBE); Susac's syndrome; Sympathetic ophthalmia; Systemic lupus erythematosus (SLE); Systemic sclerosis; Takayasu's arteritis; Temporal arteritis/Giant cell arteritis; Thrombocytopenic purpura (TTP); Tolosa-Hunt syndrome; Transverse myelitis; Type 1 diabetes; Ulcerative colitis; Undifferentiated connective tissue disease (UCTD); Uveitis; Vasculitis; Vesiculobullous dermatosis; Vitiligo; Wegener's granulomatosis (now termed Granulomatosis with Polyangiitis (GPA). Non-limiting examples of inflammatory or autoimmune diseases or conditions include infection, such as a chronic infection, latent infection, slow infection, persistent viral infection, bacterial infection, fungal infection, mycoplasma infection or parasitic infection.

In some embodiments, a library of the disclosure can comprise peptides with post-translational modifications, including, for example, acetylation, amidation, biotinylation, deamidation, farnesylation, formylation, geranylgeranylation, glutathionylation, glycation, glycosylation, hydroxylation, methylation, mono-ADP-ribosylation, myristoylation, N-acetylation, N-glycosylation, N-myristoylation, nitrosylation, oxidation, palmitoylation, phosphorylation, poly(ADP-ribosyl)ation, stearoylation, sulfation, SUMOylation, ubiquitiniation, or any combination thereof. In some embodiments, a peptide of the disclosure can comprise one or more selenocysteine residues.

In some embodiments, a peptide in a library of the disclosure is part of a protein-mRNA complex. In some embodiments, a peptide in a library of the disclosure is part of a protein-mRNA complex comprising a puromycin linkage. In some embodiments, a peptide in a library of the disclosure is part of a protein-mRNA-cDNA complex. In some embodiments, a peptide in a library of the disclosure is part of a protein-DNA complex. In some embodiments, a peptide in a library of the disclosure is part of a protein-DNA complex comprising a biotin-streptavidin linkage. In some embodiments, a peptide in a library of the disclosure is part of a protein-cDNA complex. In some embodiments, a peptide in a library of the disclosure is part of a protein-ribosome-mRNA complex. In some embodiments, a peptide in a library of the disclosure is part of a protein-ribosome-mRNA complex, where the mRNA contains a spacer sequence lacking a stop codon. In some embodiments, a peptide in a library of the disclosure is part of a protein-ribosome-mRNA-cDNA (PRMC) complex.

In some embodiments, a peptide in a library of the disclosure is purified by affinity tag purification (e.g., with a FLAG-tag). In some embodiments, a peptide in a library of the disclosure comprises a HaloTag enzymatic sequence. In some embodiments, peptides in a library of the disclosure comprise an avidin or streptavidin.

In some embodiments, a peptide in a library of the disclosure can be bound or fused to another molecule. In some embodiments, a peptide in a library of the disclosure can be bound or fused to an additional polypeptide. In some embodiments, a peptide in a library of the disclosure can be bound or fused to a polynucleic acid. In some embodiments, a peptide in a library of the disclosure can be bound or fused to a DNA. In some embodiments, a peptide in a library of the disclosure can be bound or fused to an RNA. In some embodiments, a peptide in a library of the disclosure can exist within a larger scaffold, e.g. as a part of a larger protein sequence or protein complex.

A library of the disclosure can comprise about 100, 500, 1000, 2000, 5000, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 20², 20³, 20⁴, 20⁵, 20⁶, 20⁷, 20⁸, 20⁹, 20¹⁰, 20¹¹, 20¹², 20¹³, 20¹⁴, 20¹⁵, 20¹⁶, 20¹⁷, 20¹⁸, 20¹⁹, 20²⁰, 20²¹, 20²², 20²³, 20²⁴, 20²⁵, 20²⁶, 20²⁷, 20²⁸, 20²⁹, or 20³⁰ peptides or antigens.

A library of the disclosure can comprise greater than at least about 100, 500, 1000, 2000, 5000, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 20², 20³, 20⁴, 20⁵, 20⁶, 20⁷, 20⁸, 20⁹, 20¹⁰, 20¹¹, 20¹², 20¹³, 20¹⁴, 20¹⁵, 20¹⁶, 20¹⁷, 20¹⁸, 20¹⁹, 20²⁰, 20²¹, 20²², 20²3, 20²⁴, 20²⁵, 20²⁶, 20²⁷, 20²⁸, 20²⁹, or 20³⁰ peptides or antigens.

A library of the disclosure can comprise at most about 100, 500, 1000, 2000, 5000, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 20², 20³, 20⁴, 20⁵, 20⁶, 20⁷, 20⁸, 20⁹, 20¹⁰, 20¹¹, 20¹², 20¹³, 20¹⁴, 20¹⁵, 20¹⁶, 20¹⁷, 20¹⁸, 20¹⁹, 20²⁰, 20²¹, 20²², 20²³, 20²⁴, 20²⁵, 20²⁶, 20²⁷, 20²⁸, 20²⁹, or 20³⁰ peptides or antigens.

A k-mer of the disclosure can be a 1-mer, a 2-mer, a 3-mer, a 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, or a 50-mer.

A library of the disclosure can comprise peptides with about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 constrained residues per peptide. A library of the disclosure can comprise peptides with about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 variable residues per peptide.

A library of the disclosure can comprise peptides with greater than at least about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 constrained residues per peptide. A library of the disclosure can comprise peptides with greater than at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 variable residues per peptide.

A library of the disclosure can comprise peptides with at most about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 constrained residues per peptide.

A library of the disclosure can comprise peptides with at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 600, 700, 800, 900, or 1000 variable residues per peptide.

### Peptide Library Subsets

A peptide library can be a subset of the unbiased library. In some embodiments, an algorithm can be used to select peptides in a peptide library of the disclosure. For example, an algorithm can be used to predict peptides most likely to fold or dock in an MHC/HLA binding pocket, and peptides above a certain threshold value can be selected for inclusion in the library. In some embodiments, selection of peptides for a library of the disclosure comprises prioritizing peptides based on predicted binding affinity for a certain HLA type. In some embodiments, selection of peptides for a library of the disclosure prioritizes HLA types or alleles based on prevalence in a population, e.g., a human population.

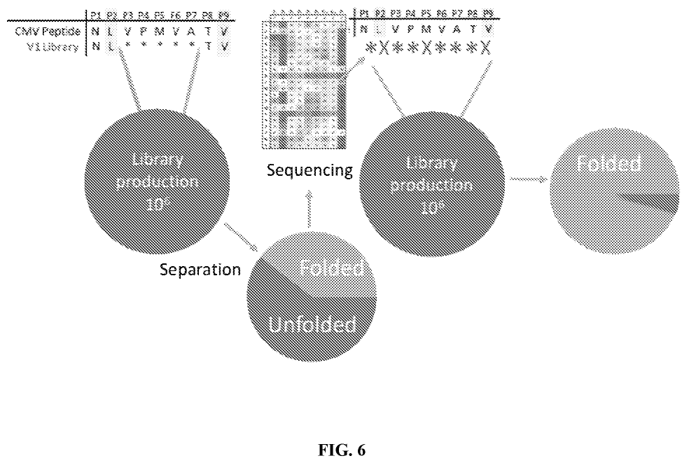

In some embodiments, a library of the disclosure comprises peptides selected based on a screening assay, for example, a functional assay for folding. An assay can be used to test for successful folding of a peptide of the disclosure. For example, a monoclonal antibody can be used in an assay to determine successful folding of a polypeptide, e.g., binding of a BB7.2 monoclonal antibody can indicate successful folding of HLA-A2. In some embodiments, correctly folded peptides are separated from misfolded peptides, sequences of the correctly folded and misfolded peptides can be determined (e.g., by sequencing identifiers disclosed herein), and a subsequent library can be enriched for correctly folded peptides (FIG. 6).

In some embodiments, a library of the disclosure comprises known, detected, or predicted neoepitopes from a cancer (e.g., colorectal cancer or non-small cell lung cancer). In some embodiments, a library of the disclosure comprises sequences associated with or enriched in a certain disease or condition.

### Antigen Libraries

This disclosure provides peptide libraries useful in a range of screening assays to identify potential diagnostic or therapeutic targets or agents. A peptide library can be an antigen library. Included in the disclosure are antigen libraries with applications in assays related to immune antigens, including T cell antigens which comprise T cell epitopes. T cell antigens and epitopes can greatly impact the function of the adaptive immune system, as they are the specific sequences that T cell receptors (TCRs) recognize. TCR recognition of cognate antigen can be important for, for example, immune recognition and effector immune responses against a pathogen, immune recognition and effector immune responses against cancer cells, and autoimmune responses (e.g., immune responses against self-tissues resulting in disease).

Antigen libraries for screening of T cell epitopes, therefore, have potential utility in, for example, research, diagnostics, treatments, and preventative measures related to infectious diseases, cancers, and autoimmune diseases. The identification of antigenic peptide sequences recognized by T cells is crucial, for example, for vaccine development (e.g., for identification of protective antigens for a given pathogen or identification of neo-antigens for use in a cancer vaccine), for cancer therapy (e.g., to identify targets for T cell-based therapies, including identification of neo-antigens), and for autoimmune research and treatments (e.g., to identify autoimmune antigens).

There are, however, a number of challenges related to the production and use of peptide libraries for applications relating to T cell antigens and epitopes. The sheer number of potentially relevant possible peptide sequences presents challenges for library production. For a peptide of 9 residues in length (a 9-mer), there are 20⁹ (approximately 5.1×10¹¹) possible sequence combinations of the 20 amino acids most commonly found in proteins. As described below, some peptides presented to T cells can be longer than 9 residues, thus the diversity of potential peptides recognized by a TCR can exceed 5.1×10¹¹. Additionally, sources of potential antigen peptides of interest (e.g., pathogens, cancer cells, tissues) can express thousands of proteins, each protein comprising a plurality of potential peptide antigens. Efficient, high-throughput methods are required to generate any library of such diversity.

Additional challenges relate to the way peptides must be presented to TCR to initiate binding of sufficient affinity to be useful in an experimental or therapeutic context. In vivo, TCRs recognize peptides presented by major histocompatibility complex (MHC) molecules. In order to bind TCR, peptide antigens of a peptide library must also be presented in the context of MHC. Additionally, in order to achieve binding of sufficient affinity as to be useful in an experimental or therapeutic context, a multimer of peptide-MHC (pMHC) complexes are required. Traditional methods to generate pMHC multimers are low-throughput and labor-intensive, and the resulting polypeptides are prone to misfolding and poor loading of peptide antigen.

Provided herein are libraries of diverse pMHC multimers and methods for their production, including high-throughput methods. In some embodiments, the pMHC multimers further comprise nucleic acid identifiers, allowing for convenient detection and quantification of binding as described elsewhere herein.

pMHC Multimers

Peptides are presented to TCRs by two general classes of MHC, MHC class I (MHC-I) and MHC class II (MHC-II). In humans, MHC is also referred to as human leukocyte antigen (HLA). The genes encoding HLA are highly variable between different individuals, and different HLA genes can have different characteristics with regards to peptide binding and antigen presentation. Humans have three main MHC class I genes, known as HLA-A, HLA-B, and HLA-C. The proteins produced from these genes are present on the surface of almost all cells. Additional, non-classical class I genes include HLA-E, HLA-F, and HLA-G. There are six main MHC class II genes in humans: HLA-DPA1, HLA-DPB1, HLA-DQA1, HLA-DQB1, HLA-DRA, and HLA-DRB1. There are multiple allelic forms of many MHC genes, each of which can present a plurality of peptides in a shallow antigen-binding groove generated by two antiparallel α-helices sitting on top of a β-sheet. Peptides presented by MHC-I can be bound in an extended conformation with both N and C termini bound inside a closed groove, limiting their size (for example, to 8-10 residues). Peptides presented by MHC-II can also be bound in an extended conformation, but, because of the open nature of the groove, can be longer (for example, 14-20 residues).

To generate pMHCs, constructs for expression of the subunits can be generated. For example, constructs for expression of the MHC-I heavy chain and beta-2-microglobulin (β2m) can be generated. The transmembrane domain of the heavy chain can be genetically deleted to facilitate purification, and a biotin recognition site added to the C-terminus. The heavy chain, β2m, and peptide can each be expressed, for example, recombinantly expressed as inclusion bodies in E. coli cultures, or expressed in eukaryotic cells, such as insect or mammalian cells. The biotin recognition site can be biotinylated, for example, using the BirA enzyme. The heavy chain, β2m, and peptide can then be treated with a denaturant, re-folded into pMHC monomers, and purified by size exclusion chromatography. MHC molecules that do not correctly associate with a peptide can be unstable, dissociate, and misfold. A similar technique can be used to generate peptide-MHC-II complexes (pMHC-II).

MHC monomers can then be multimerized, to form, for example, dimers, tetramers, pentamers, octomers, streptamers, or dextramers. Dimers can be produced by genetic fusion of the extracellular domain of an MHC molecule, for example, as a fusion with an immunoglobulin scaffold that binds a second MHC. Tetramers can be generated, for example, by the addition of a streptavidin or avidin ‘backbone’ to MHC monomers with biotinylated C-termini. Alternatively, Streptavidin domains can be expressed as a C-terminal fusion to an MHC chain, facilitating self-assembly into tetramers. MHC Tetramers and octomers can also be generated by introducing a point mutation into a free cysteine at the C terminus of an MHC chain, which can be alkylated with biotin containing iodoacetamide or maleimide derivatives. Streptavidin conjugates can be used for oligomerization. By using a branched peptide containing one biotin and two maleimide moieties (DMGS), this strategy allows the preparation of octameric MHC complexes. MHC pentamers can be generated by complexing five MHC monomers via a self-assembling coiled-coil domain. MHC dextramers can be generated by attaching a plurality of MHC complexes, e.g. ten or more, to a dexran polymer backbone. MHC streptamers can be generated by linking biotinylated C termini of MHC chains to a multimerized strep-Tactin or Strep-tag backbone, leading to a complex comprising 8-12 MHC monomers.

Single-Chain pMHC

MHC molecules can be engineered as single chain peptide-MHC polypeptides (sc-pMHC) that comprise the subunits of an assembled pMHC. Such sc-pMHC can, for example, simplify loading of a peptide antigen in to the MHC binding groove. Such a sc-pMHC can also, for example, promote efficient loading of the linked peptide antigen compared to other, contaminating peptides that can occupy the binding groove. Sc-pMHC multimers can exhibit specific and dose-dependent binding to antigen-specific T cells (FIG. 12).

A sc-pMHC can comprise an antigenic peptide, a heavy chain and/or a beta-2-microglobulin (β2m), corresponding to MHC-I. An MHC-I sc-pMHC can comprise an antigenic peptide and a heavy chain. An MHC-I sc-pMHC can comprise an antigenic peptide and a β2m. An MHC-I sc-pMHC can comprise an antigenic peptide, a beta-2-microglobulin (β2m), and a heavy chain. In some embodiments, a MHC-I sc-pMHC can comprise a single polypeptide with an antigenic peptide and a flexible linker that connects the antigenic peptide to the heavy chain. In some embodiments, the MHC-I sc-pMHC can further comprise another flexible linker that connects β2m to either the antigenic peptide or to the heavy chain. In some embodiments, an MHC-I sc-pMHC can comprise a single polypeptide with an antigenic peptide and a flexible linker that connects the antigenic peptide to β2m. In some embodiments, the MHC-I sc-pMHC can further comprise another flexible linker that connects a heavy chain to either the antigenic peptide or to β2m.

A sc-pMHC can comprise an antigenic peptide, alpha chain and/or beta chain, corresponding to MHC-II. An MHC-II sc-pMHC can comprise an antigenic peptide and an alpha chain. An MHC-II sc-pMHC can comprise an antigenic peptide and a beta chain. An MHC-II sc-pMHC can comprise an antigenic peptide, an alpha chain, and a beta chain. An MHC-II sc-pMHC can comprise a single polypeptide with an antigenic peptide and a flexible linker that connects the antigenic peptide to the beta chain. In some embodiments, the MHC-II sc-pMHC can further comprise another flexible linker that connects the alpha chain to either the antigenic peptide or the beta chain. In some embodiments, an MHC-II sc-pMHC can comprise a single polypeptide with an antigenic peptide and a flexible linker that connects the antigenic peptide to the alpha chain. In some embodiments, the MHC-II sc-pMHC can further comprise another flexible linker that connects the beta chain to either the antigenic peptide or the alpha chain.

A sc-pMHC can be in any order, e.g., peptide at the C-terminus of the single polypeptide to allow for greater diversity at the N-terminus of the antigenic peptide. Other mechanisms may be employed to generate greater diversity at the N-terminus if the antigenic peptide is at the N-terminus, e.g., enzymatic cleavage of the N-terminus. sc-pMHC monomers can be multimerized as described for MHC monomers above, e.g., into dimers, tetramers, pentamers, octomers, streptamers, or dextramers.

In some embodiments, a library of the disclosure comprises a plurality of sc-pMHCs. In some embodiments, a library of the disclosure comprises a plurality of sc-pMHC, wherein k-mer sequences are localized within the antigenic peptide part of the sc-pMHC.

Peptide-MHC multimers or antibodies that specifically bind pMHC multimers can be conjugated with a fluorescent label, allowing for identification of T cells that bind the peptide-MHC multimer, for example, via flow cytometry or microscopy. T cells can also be selected based on a fluorescence label through, e.g., fluorescence activated cell sorting. However, a limitation to this approach relates to the number of different fluorescence labels and detectors available, which limits use of fluorescent-based methods for high throughput antigen library screens.

In some embodiments, a library of the disclosure comprises sc-pMHC multimers conjugated with nucleotide identifiers as described elsewhere, allowing for convenient detection and quantification of antigen-specific binding to TCR. Such a library can allow, for example, detection of T cells specific for a given antigen, multiplex detection of T cell specificities in a given sample, matching of TCR sequence with specificity (e.g., via single cell sequencing), comparative TCR affinity determination, determination of a consensus specificity sequence of a given TCR, or mapping of antigen responsiveness of T cells against sequences of interest.

### Linkers

In some embodiments, the disclosure provides polypeptide sequences wherein two polypeptide sequences or domains are joined by a linker. In some embodiments, the disclosure provides polypeptide sequences wherein three or more polypeptide sequences or domains are joined by linkers. In some embodiments, the disclosure provides polypeptide sequences wherein four or more polypeptide sequences or domains are joined by linkers. In some embodiments, the disclosure provides polypeptide sequences wherein five or more polypeptide sequences or domains are joined by linkers. In some embodiments, the disclosure provides polypeptide sequences wherein six or more polypeptide sequences or domains are joined by linkers.

A linker may be a chemical bond, e.g., one or more covalent bonds or non-covalent bonds. In some embodiments, the linker is a covalent bond. In some embodiments, the linker is a non-covalent bond. In some embodiments, a linker is a peptide linker. Such a linker may be between 2-30 amino acids, or longer. In some embodiments, a linker can be used, e.g., to space the polypeptide sequences or domains from one another. In some embodiments, a linker can be positioned between domains, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release the polypeptide sequences or domains from each other, but sufficiently stable to prevent premature cleavage.

Flexible peptide linkers can have sequences consisting primarily of stretches of Gly and Ser residues (“GS” linker). Flexible peptide linkers can be useful for joining domains that require a certain degree of movement or interaction and may include small, non-polar (e.g., Gly) or polar (e.g., Ser or Thr) amino acids. Incorporation of Ser or Thr can promote the stability of the linker in aqueous solutions by forming hydrogen bonds with the water molecules, thereby reducing unfavorable interactions between the linker and protein moieties. Flexible linkers can include, for example, single copies or repeats of any of the sequences listed in TABLE 1.

TABLE 1

| SEQ | |
|---|---|
| ID NO: | Sequence |
| 1 | GGGGS |
| 2 | GGGGGG |
| 3 | GGGGGGGG |
| 4 | KESGSVSSEQLAQFRSLD |
| 5 | EGKSSGSGSESKST |
| 6 | GSAGSAAGSGEF |

Rigid linkers can be useful to keep a fixed distance between domains. Rigid linkers can also be useful when a spatial separation of domains is critical to preserve the stability or bioactivity of one or more components. Rigid linkers can have an alpha helix-structure or Pro-rich sequence. Rigid linkers can, for example, comprise the sequences (EAAAK)ₙ, A(EAAAK)ₙA (XP)ₙ, with n representing any number of repeats (e.g., 2-5), and X representing any amino acid (e.g., Ala, Lys, or Glu).

A cleavable peptide linker can be used to allow removal or release of a polypeptide sequence or domain. In some embodiments, a linker can be cleaved under specific conditions, such as the presence of reducing reagents or enzymes. In vivo cleavage of linkers in fusions may also be carried out by enzymes or proteases that are expressed in vivo under certain conditions, in specific cells or tissues, or constrained within certain cellular compartments. Specificity of many enzymes or proteases can offer slower cleavage of the linker in constrained compartments. For example, a cleavable linker can comprise a cleavage sequence for a matrix metalloproteinase (MMP) or another protease. Another example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369.

In some embodiments, a linker can be a peptide bond, for example the C-terminus of a peptide sequence or domain can be fused to the N-terminus of another peptide sequence or domain via a peptide bond.

Further examples of linkers include hydrophobic linkers, such as a negatively charged sulfonate groups; lipids, such as a poly (—CH2-) hydrocarbon chains, such as polyethylene glycol (PEG) groups, unsaturated variants thereof, hydroxylated variants thereof, amidated or otherwise N-containing variants thereof; noncarbon linkers; carbohydrate linkers; phosphodiester linkers, or other molecule capable of covalently linking two or more polypeptides. Non-covalent linkers can also be used, e.g., biotin-streptavidin linkers.

### Peptide Identifiers

This disclosure provides peptide libraries, including, for example, high diversity peptide libraries useful in a range of therapeutic and diagnostic screens. The peptide libraries provided can be used, for example, to screen for disease-specific or organ-specific peptides, to screen for peptides with therapeutic applications, to screen for peptides with diagnostic applications, to screen for tumor-targeting peptides, to screen for antibody epitopes or antigens, to screen for T cell epitopes or antigens, to screen for antimicrobial peptides, or any combination thereof.

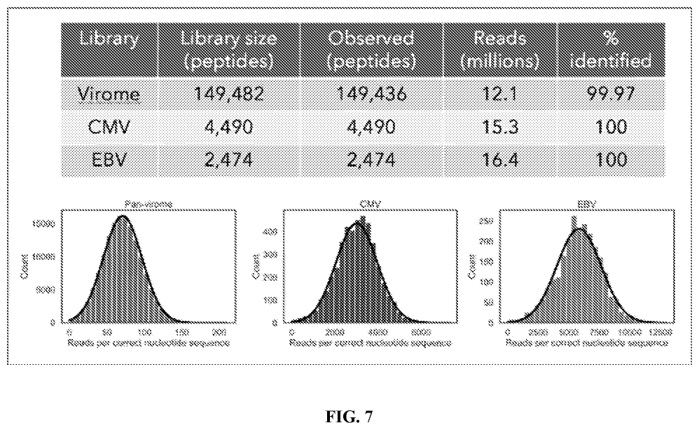

Assays utilizing peptide libraries can be limited by methods required to detect and quantify individual peptides. Nucleic acid identifiers can be used to tag each peptide in a library with a unique nucleic acid sequence, allowing detection and quantification of individual peptides using nucleic acid-based methods, for example, PCR amplification or DNA sequencing. A nucleic acid identifier can be a unique nucleic acid sequence. In some embodiments, nucleic acid identifiers allow detection and quantification of individual peptides when a plurality of peptides are pooled in a common experimental condition. In some embodiments, nucleic acid identifiers allow detection and quantification of individual peptides when all peptides of a peptide library are pooled in a common experimental condition. In some embodiments, nucleic acid identifiers can be used to tag nucleic acids (e.g., DNA, mRNA, cDNA) in an experimental condition, for matching to a peptide of a peptide library present in the same experimental condition and having the same nucleic acid identifier. In some embodiments, nucleic acid identifiers allow validation of library diversity, wherein the nucleic acid identifiers are subjected to DNA sequencing, and observed reads are mapped to predicted library sequences to identify the presence or absence of peptides in the library (FIG. 7). In some embodiments, identifiers are additionally used to normalize read-based quantification. In some embodiments, an identifier is a self-identifier that corresponds to all or a part of a nucleic acid sequence that encodes the peptide that it identifies. In some embodiments, an identifier is not a self-identifier (e.g., does not contain a nucleic acid sequence that encodes the peptide it identifies).

An identifier can have any nucleic acid sequence. In some embodiments, an identifier can be a single stranded or double stranded DNA polynucleotide. In some embodiments, an identifier can be an RNA polynucleotide. In some embodiments, an identifier can be a hybrid DNA and RNA polynucleotide. In some embodiments, an identifier can comprise synthetic or chemically modified nucleotides or conjugates (e.g., to enhance stability or facilitate attachment to a peptide).

Nucleic acid identifiers can be attached to peptides covalently or non-covalently.

Proteins or peptides can have the identifier attached by an in vitro translation method to generate protein-mRNA complexes, protein-mRNA-cDNA complexes, protein-DNA complexes, protein-cDNA complexes, protein-ribosome-mRNA complexes, or protein-ribosome-mRNA-cDNA (PRMC) complexes, which can contain synthetic identifiers at the 5′ end of protein open reading frames (ORFs). In some embodiments, mRNA display can be performed using mRNA templates comprising puromycin, and an in vitro translation (IVT) system comprising purified components. mRNA-protein complexes bearing proteins of interest can be enriched by affinity tag purification, for example, FLAG-tag purification. In some embodiments, ribosome display can be performed using mRNA templates comprising spacer sequences lacking stop codons, and an in vitro translation (IVT) system comprising purified components. Protein-ribosome-mRNA complexes bearing proteins of interest can be enriched by affinity tag purification, for example, FLAG-tag purification. In some embodiments, ribosome display can be performed by using mRNA-cDNA hybrids as templates, and an in vitro translation (IVT) system comprising purified components. PRMC complexes bearing proteins of interest can be enriched by affinity tag purification, for example, FLAG-tag purification. In some embodiments, DNA display can be performed by using biotinylated DNA templates, and an in vitro transcription and translation system comprising purified components. Protein-DNA complexes can form via biotin-streptavidin binding, and protein-DNA complexes can be enriched by affinity tag purification, for example, FLAG-tag purification. In some embodiments, mRNA is synthesized from DNA as part of an in vitro transcription and translation system. In some embodiments, cDNA is reverse transcribed from mRNA before or after purification of a complex comprising peptide and mRNA.

An identifier can be attached to a protein or peptide enzymatically. For example, a fusion protein can be generated comprising a HaloTag enzymatic sequence, and the enzymatic activity can covalently conjugate the fusion protein to a HaloTag ligand-modified double stranded DNA.

An identifier can be attached to a protein or peptide using non-covalent interactions. For example, biotinylated polynucleotide identifiers can bind to an avidin or streptavidin associated with the peptide. The avidin or streptavidin can be a part of the peptide sequence, or can be associated with it in some other way, e.g., a streptavidin backbone used in assembly of a protein multimer.

In some embodiments, a unique identifier can be used for each unique peptide in a library. In some embodiments, identifiers can be shared between two or more peptides in a peptide library, e.g. when the peptides comprise the same sequence. In some embodiments, identifiers can comprise a sequence that is shared between multiple or all peptides of a library, and a sequence that is unique to a peptide in a library. In some embodiments, identifiers can comprise one or more sequence portions that are shared between multiple or all peptides of a library, and one or more other sequence portions that are unique to a peptide in the library. In some embodiments, a sequence shared between identifiers can be used for identifier amplification (e.g., PCR amplification with suitable primers). In some embodiments, a sequence unique to one identifier or shared between a subset of identifiers can be used for detection or quantification via qPCR (e.g., sequences for hydrolysis probes, such as TaqMan probes). In some embodiments, a sequence unique to one identifier or shared between a subset of identifier can be used for detection or quantification via sequencing.

In some embodiments, nucleic acid identifiers can comprise sequences that code for amino acids. In some embodiments, two or more nucleic acid identifiers can comprise different sequences that code for the same amino acids, (e.g., use different codons). In some embodiments, differential codon utilization in nucleic acid identifiers can allow additional information to be stored in identifiers.

In some embodiments, an identifier can comprise a unique, in silico-generated sequence; each identifier sequence can be assigned to its peptide comprising the unique sequence in the library and the identifier-peptide assignment can be stored in a database. In some embodiments, an identifier can comprise a nucleotide sequence that codes for all or part of the peptide it identifies. In some embodiments, an identifier can comprise a nucleotide sequence that codes for an open reading frame. In some embodiments, an identifier can comprise a nucleotide sequence that includes a promoter sequence. In some embodiments, an identifier can comprise a nucleotide sequence that includes a binding site for a DNA-binding protein, e.g. a transcription factor or polymerase enzyme. In some embodiments, an identifier can comprise one or more sequences targeted by a nuclease, e.g., a restriction enzyme. In some embodiments, an identifier can comprise at least one sequence element necessary for in vitro transcription and translation of a sequence.

In some embodiments, an identifier can comprise a nucleotide sequence that codes for a Major histocompatibility complex (MHC) molecule or fragment thereof. In some embodiments, an identifier can comprise a nucleotide sequence that encodes a single chain peptide-MHC polypeptide (sc-pMHC) or fragment thereof. In some embodiments, an identifier can comprise a nucleotide sequence that codes for the antigenic peptide of a sc-pMHC.

In some embodiments, an identifier can be part of a protein-mRNA complex. In some embodiments, an identifier can be part of a protein-mRNA complex comprising a puromycin linkage. In some embodiments, an identifier can be part of a protein-mRNA-cDNA complex. In some embodiments, an identifier can be part of a protein-DNA complex. In some embodiments, an identifier can be part of a protein-DNA complex comprising a biotin-streptavidin linkage. In some embodiments, an identifier can be part of a protein-cDNA complex. In some embodiments, an identifier can be part of a protein-ribosome-mRNA complex. In some embodiments, an identifier can be part of a protein-ribosome-mRNA complex, where the mRNA contains a spacer sequence lacking a stop codon. In some embodiments, an identifier can be part of an mRNA-cDNA hybrid. In some embodiments, an identifier can be part of a PRMC complex.

In some embodiments, an identifier can comprise a HaloTag ligand, e.g., a chloroalkane linker bound to a functional group, such as biotin or a fluorescent dye.

In some embodiments, an identifier can comprise a biotinylated nucleotide sequence. In some embodiments, an identifier can be biotinylated by PCR amplification with a biotinylated primer(s). In some embodiments, an identifier can be biotinylated by enzymatic incorporation of a biotinylated label, e.g., a biotin dUTP label, by use of Klenow DNA polymerase enzyme, nick translation or mixed primer labeling RNA polymerases, including T7, T3, and SP6 RNA polymerases. In some embodiments, an identifier can be biotinylated by photobiotinylation, e.g., photoactivatable biotin can be added to the sample, and the sample irradiated with UV light.

In some embodiments, an identifier can be generated from a template polynucleotide, e.g., via PCR amplification of a template DNA. In some embodiments, an identifier can be generated de novo, e.g., via chemical synthesis, solid-phase DNA synthesis, column-based oligonucleotide synthesis, microarray-based oligonucleotide synthesis, or other synthetic methods. In some embodiments, a template polynucleotide can comprise a nucleotide sequence that codes for an open reading frame. In some embodiments, a template polynucleotide can comprise a nucleotide sequence that includes a promoter sequence. In some embodiments, a template polynucleotide can comprise a nucleotide sequence that includes a binding site for a DNA-binding protein, e.g., a transcription factor or polymerase enzyme. In some embodiments, a template polynucleotide can comprise one or more sequences targeted by a nuclease, e.g., a restriction enzyme. In some embodiments, a template polynucleotide can comprise all sequence elements necessary for in vitro transcription and translation of a sequence. In some embodiments, a template polynucleotide does not comprise all sequence elements necessary for in vitro transcription and translation of a sequence. In some embodiments, template polynucleotides encoding two or more nucleic acid identifiers can comprise different sequences that code for the same amino acids, (e.g., use different codons). In some embodiments, differential codon utilization in template polynucleotides can be used to identify a class or subclass of peptides within a library. In some embodiments, differential codon utilization in nucleic acid identifiers can allow additional information to be stored in a template polynucleotide.

An identifier of the disclosure can be any length. In some embodiments, an identifier can be about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 320, 340, 360, 380, 400, 420, 440, 460, 480, 500, or greater nucleotides in length.

In some embodiments, an identifier can be at greater than at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 320, 340, 360, 380, 400, 420, 440, 460, 480 or 500 nucleotides in length.

In some embodiments, an identifier can be at most about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 320, 340, 360, 380, 400, 420, 440, 460, 480, 500, or greater nucleotides in length.

In some embodiments, an identifier can be in the range of about 4-500 nucleotides in length. In some embodiments, an identifier can be in the range of about 25-500 nucleotides in length. In some embodiments, an identifier can be in the range of about 27-300 nucleotides in length. In some embodiments, an identifier can be in the range of about 27-120 nucleotides in length. In some embodiments, an identifier can be in the range of about 50-120 nucleotides in length. In some embodiments, an identifier can be in the range of about 80-120 nucleotides in length. In some embodiments, an identifier can be in the range of about 40-50 nucleotides in length. In some embodiments, an identifier can be in the range of about 5-15 nucleotides in length. In some embodiments, an identifier can be in the range of about 6-10 nucleotides in length.

### Synthesis Methods

Peptides of a peptide library can be synthesized via chemical methods, for example, tea bag synthesis, digital photolithography, pin synthesis, and SPOT synthesis. For example, an array of peptides can be generated via SPOT synthesis, where amino acid chains are built on a cellulose membrane by repeated cycles of adding amino acids, and cleaving side-chain protection groups.

Biological peptide libraries, such as phage display, bacterial display, or yeast display, involve fusion peptides rather than isolated molecules. Biological peptide libraries can be subject to diversity limitations and redundancy. The presence of phage or bacterial components can lead to confounding effects, for example, binding of assay components to bacterial components rather than the peptide of interest, or in an assay comprising cells, immune activation via innate immune mechanisms.

Peptides can be expressed using recombinant DNA technology, for example, introducing an expression construct into bacterial cells, insect cells, or mammalian cells, and purifying the recombinant protein from cell extracts. Recombinant proteins produced in this manner, however, often express insolubly, as soluble aggregates, may be proteolysed or can be undetectable in cell extracts.

Peptides can be synthesized by in vitro transcription and translation, where synthesis utilizes the biological principles of transcription and translation in a cell-free context, for example, by providing a nucleic acid template, relevant building blocks (e.g., RNAs, amino acids), enzymes (e.g., RNA polymerase, ribosomes), and conditions. In vitro transcription and translation can include cell-free protein synthesis (CFPS). The peptide can be synthesized using the IVTT system that can both transcribe, for example, a DNA construct into RNA, and then translate the RNA into a protein. In some embodiments, a DNA or RNA construct comprises a puromycin. In some embodiments, a DNA or RNA construct comprises a spacer sequence lacking a stop codon.

In some embodiments, a nucleic acid of the disclosure can be generated de novo, e.g., via chemical synthesis, solid-phase DNA synthesis, column-based oligonucleotide synthesis, microarray-based oligonucleotide synthesis, or other synthetic methods. In some embodiments, a nucleic acid of the disclosure can be generated from a template polynucleotide, e.g., via PCR amplification of a template DNA.

A nucleotide sequence encoding a methionine residue at the N-terminus of the peptide and a cleavable moiety can be encoded in the DNA construct or RNA construct. The cleavable moiety is situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using an enzyme, e.g., a protease, specific to the cleavable moiety, which can also cleave off the cleavable moiety from the remainder of the peptide.

An example of a cleavable moiety that can be encoded in a DNA or RNA construct as described herein includes any cleavable moiety cleaved by an enzyme. In some embodiments, a cleavable moiety can be cleaved by a protease. The cleavage moiety can be cleaved off of the peptide using an enzyme specific for the cleavage moiety. The enzyme can be, for example, Factor Xa, human rhinovirus 3C protease, AcTEV™ Protease, WELQut Protease, Genenase™ small ubiquitin-like modifier (SUMO) protein, Ulp1 protease, or enterokinase. The Ulp1 protease can cleave off a cleavage moiety in a specific manner by recognizing the tertiary structure, rather than an amino acid sequence. Enterokinase (enteropeptidase) can also be used to cleave the cleavage moiety from the candidate peptide. Enterokinase can cleave after lysine at the following cleavage site: Asp-Asp-Asp-Asp-Lys (SEQ ID NO.: 7). Enterokinase can also cleave at other basic residues, depending on the sequence and conformation of the protein substrate.

After translation of the construct encoding the peptide, an N-terminus amino acid residue can be cleaved to produce the peptide for the high diversity peptide library. In some embodiments, at least one N-terminus amino acid residue is cleaved to produce the peptide. In some embodiments, one or more N-terminus amino acids are cleaved, such as 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 140, 150, 160, 170, 180, 190, 200, 250 or more, N-terminus amino acid residues are cleaved to produce the peptide. The N-terminus amino acid can be any amino acid residue. The N-terminus amino acid residue can be a methionine amino acid residue.

### Methods of Making and Using Peptide Libraries

The peptide libraries described herein can be used in various assays.

In some embodiments, the peptide libraries described herein can be used for isolating cell-peptide pairs. In some embodiments, the method for isolating cell-peptide pairs comprises contacting a plurality of cells with a peptide library, wherein the peptide library has a diversity of more than 10, more than 100, more than 500, more than 1000, more than 2,000, more than 5,000, more than 10,000, more than 10⁶, more than 10⁷, more than 10⁸, more than 10⁹, or more than 10¹⁰ unique peptides; and generating a plurality of compartments, wherein a compartment of the plurality comprises a cell of the plurality of cells bound to a peptide of the peptide library, thereby isolating the cell-peptide pair in the compartment. In some embodiments, a peptide library is used for peptide library evaluation for isolating cell-peptide pairs after de novo target discovery. The cell-peptide pair can be a receptor-ligand pair. The cell-peptide pair can be a TCR-antigen pair. The cell-peptide pair can be a BCR-antigen pair. In some embodiments, a cell can be transfected or transduced to express a receptor. In some embodiments, a cell can be transfected or transduced to express a TCR. In some embodiments, a cell can be transfected or transduced to express a BCR. In some embodiments, a non-lymphocyte cell can be transfected or transduced to express TCR. In some embodiments, a non-lymphocyte cell can be transfected or transduced to express a BCR. In some embodiments, a peptide of the plurality of peptides comprises an identifier as described herein. A compartment can be a separate space, e.g., a well, a plate, a divided boundary, a phase shift, a vessel, a vesicle, a cell, etc.

The methods and compositions described herein can be used for identifying a cell-peptide pair. In some embodiments, a method of identifying a cell-peptide pair comprises contacting a plurality of cells with a library of peptides wherein the library of peptides has a diversity of more than more than 10, more than 100, more than 500, 1000, more than 2,000, more than 5,000, more than 10,000, more than 10⁶, more than 10⁷, more than 10⁸, more than 10⁹, or more than 10¹⁰ unique peptides; compartmentalizing a cell of the plurality of cells bound to a peptide of the library of peptides in a single compartment, wherein the peptide comprises a unique peptide identifier; and determining the unique peptide identifier for each peptide bound to the compartmentalized cell. In some embodiments, a peptide library is used for peptide library evaluation for identifying a cell-peptide pairs after de novo target discovery. The cell-peptide pair can be a receptor-ligand pair. The cell-peptide pair can be a TCR-antigen pair. The cell-peptide pair can be a BCR-antigen pair. In some embodiments, a cell can be transfected or transduced to express a receptor. In some embodiments, a cell can be transfected or transduced to express a TCR. In some embodiments, a cell can be transfected or transduced to express a BCR. In some embodiments, a non-lymphocyte cell can be transfected or transduced to express TCR. In some embodiments, a non-lymphocyte cell can be transfected or transduced to express a BCR. In some embodiments, the peptide library is an antigen library. The plurality of peptides can be a plurality of antigens. The plurality of peptides can be a plurality of pMHC multimers as described herein. The plurality of peptides can be a plurality of sc-pMHCs as described herein. In some embodiments, a peptide of the plurality of peptides comprises an identifier as described herein.

In some embodiments, the peptide libraries described herein can be used for isolating lymphocyte-peptide pairs. In some embodiments, the method for isolating lymphocyte-peptide pairs comprises contacting a plurality of lymphocytes with a peptide library, wherein the peptide library has a diversity of more than 10, more than 100, more than 500, more than 1000, more than 2,000, more than 5,000, more than 10,000, more than 10⁶, more than 10⁷, more than 10⁸, more than 10⁹, or more than 10¹⁰ unique peptides; and generating a plurality of compartments, wherein a compartment of the plurality comprises a lymphocyte of the plurality of lymphocytes bound to a peptide of the peptide library, thereby isolating the lymphocyte-peptide pair in the compartment. In some embodiments, a peptide library is used for peptide library evaluation for isolating lymphocyte-peptide pairs after de novo target discovery. The lymphocyte can be a T cell, B cell, or NK cell. In some embodiments, the peptide library is an antigen library. The plurality of peptides can be a plurality of antigens. The plurality of peptides can be a plurality of pMHC multimers as described herein. The plurality of peptides can be a plurality of sc-pMHCs as described herein. The lymphocyte-peptide pair can be a TCR-antigen pair. The lymphocyte-peptide pair can be a BCR-antigen pair. In some embodiments, a peptide of the plurality of peptides comprises an identifier as described herein. A compartment can be a separate space, e.g., a well, a plate, a divided boundary, a phase shift, a vessel, a vesicle, a cell, etc.

The methods and compositions described herein can be used for identifying a lymphocyte-peptide pair. In some embodiments, a method of identifying a lymphocyte-peptide pair comprises contacting a plurality of lymphocytes with a library of peptides wherein the library of peptides has a diversity of more than more than 10, more than 100, more than 500, 1000, more than 2,000, more than 5,000, more than 10,000, more than 10⁶, more than 10⁷, more than 10⁸, more than 10⁹, or more than 10¹⁰ unique peptides; compartmentalizing a lymphocyte of the plurality of lymphocytes bound to a peptide of the library of peptides in a single compartment, wherein the peptide comprises a unique peptide identifier; and determining the unique peptide identifier for each peptide bound to the compartmentalized lymphocyte. In some embodiments, a peptide library is used for peptide library evaluation for identifying lymphocyte-peptide pairs after de novo target discovery. The lymphocyte can be a T cell, B cell, or NK cell. In some embodiments, the peptide library is an antigen library. The plurality of peptides can be a plurality of antigens. The plurality of peptides can be a plurality of pMHC multimers as described herein. The plurality of peptides can be a plurality of sc-pMHCs as described herein. In some embodiments, a peptide of the plurality of peptides comprises an identifier as described herein. A lymphocyte-peptide pair can be a TCR-antigen pair. A lymphocyte-peptide pair can be a BCR-antigen pair.

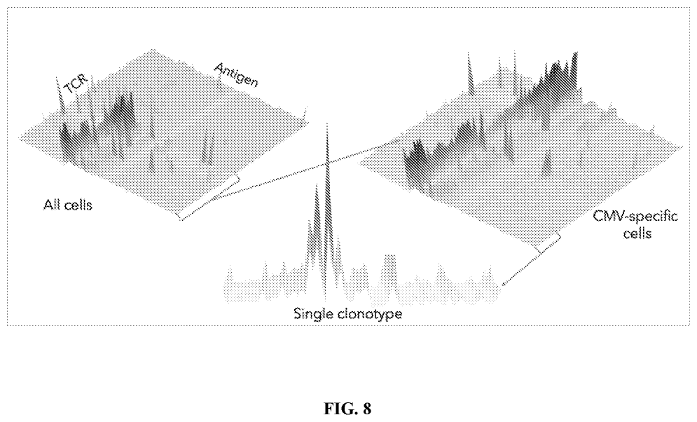

In some embodiments, the compositions and methods disclosed herein can be used to identify a plurality of peptides, ligands, agonists, antagonists, antigens, or epitopes that bind to a receptor, immune receptor, TCR, BCR, or antibody. In some embodiments, the compositions and methods disclosed herein can be used to identify a plurality of receptors, immune receptors, TCRs, BCRs, or antibodies that bind a peptide. In some embodiments, the compositions and methods disclosed herein can be used to identify a plurality of receptors, immune receptors, TCRs, BCRs, or antibodies that bind a plurality of peptides, ligands, agonists, antagonists, antigens, or epitopes (for example, a plurality of TCRs that bind to antigens in a pathogen library (FIG. 8), cancer library, or autoimmune library).

In some embodiments, the compositions and methods disclosed herein are used for identifying receptor-ligand specificity. In some embodiments, the compositions and methods disclosed herein are used for identifying receptor-agonist specificity. In some embodiments, the compositions and methods disclosed herein are used for identifying receptor-antagonist specificity. In some embodiments, the compositions and methods disclosed herein are used for identifying immune receptor-antigen specificity (e.g., TCR-antigen specificity, BCR-antigen-specificity). In some embodiments, the compositions and methods disclosed herein are used for identifying antibody-antigen-specificity.

In some embodiments, the identity of a receptor, immune receptor, TCR, BCR, or antibody of the disclosure is determined by sequencing (e.g., sequencing a variable, hypervariable region or complementarity determining region (CDR) of a TCR, BCR, or antibody). In some embodiments, a CDR1, CDR2, or CDR3 sequence is identified for a TCR alpha chain, TCR beta chain, TCR gamma chain, TCR delta chain, antibody heavy chain, or antibody light chain.

In some embodiments, the identity of a peptide, ligand, agonist, antagonist, antigen, or epitope is determined by sequencing (e.g., using an identifier as disclosed herein).

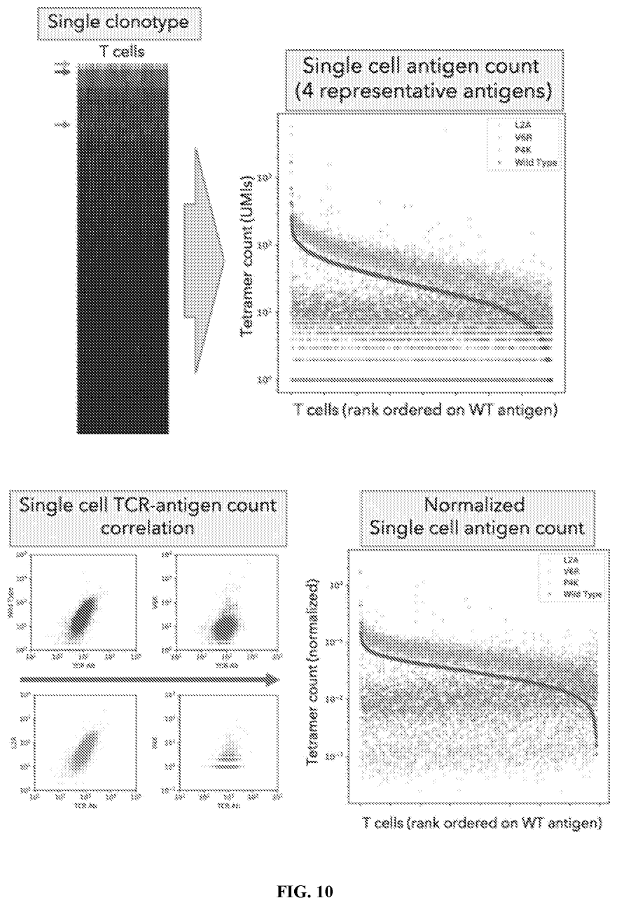

In some embodiments, the compositions and methods disclosed herein are used to identify peptides, antigens, or epitopes that bind a TCR. In some embodiments, the compositions and methods disclosed herein are used to determine how mutations in an identified peptide, antigen, or epitope affect TCR binding (FIG. 10). In some embodiments, the compositions and methods disclosed herein are used to identify mutations in an identified peptide, antigen, or epitope that result in enhanced or reduced TCR binding affinity. In some embodiments, the compositions and methods disclosed herein are used to identify mutations in an identified peptide, antigen, or epitope that retain TCR binding affinity. In some embodiments, the compositions and methods disclosed herein are used to identify mutations in an identified peptide, antigen, or epitope that result in loss of TCR binding affinity.

In some embodiments, the compositions and methods disclosed herein are used to determine how mutations in a receptor, immune receptor, TCR, BCR, or antibody identified using the methods described herein alter the binding of a peptide, ligand, agonist, antagonist, antigen, or epitope. In some embodiments, the compositions and methods disclosed herein are used to identify mutations in an identified receptor, immune receptor, TCR, BCR, or antibody that result in decreased or increased binding affinity for a peptide, ligand, agonist, antagonist, antigen, or epitope. In some embodiments, the compositions and methods disclosed herein can be used to identify mutations in an identified receptor, immune receptor, TCR, BCR, or antibody that retain binding of a peptide, ligand, agonist, antagonist, antigen, or epitope. In some embodiments, the compositions and methods disclosed herein can be used to identify mutations in an identified receptor, immune receptor, TCR, BCR, or antibody that result in loss of binding of a peptide, ligand, agonist, antagonist, antigen, or epitope.

In some embodiments, the compositions and methods disclosed herein can be used to identify a TCRs among a population of diverse TCRs that binds a given peptide, antigen, or epitope of a peptide library. The identified TCRs can then be compared to identified TCRs from samples from different subjects or from different samples from the same subject (e.g., samples from different tissues).

In some embodiments, the methods disclosed herein are performed on T cells from a plurality of subjects. In some embodiments, analysis of data from multiple subjects allows identification of antigens recognized by multiple subjects. In some embodiments, analysis of data from multiple subjects allows identification of antigens recognized by multiple TCR clonotypes. In some embodiments, analysis of data from multiple subjects allows identification of antigens recognized by multiple patients, e.g., multiple cancer patients, multiple patients with an autoimmune condition, or multiple patients with protective immunity against a pathogen. In some embodiments, analysis of data from multiple subjects allows identification of antigens recognized in subjects comprising different HLA types or alleles. In some embodiments, analysis of data from multiple subjects allows identification of distinct hypervariable or complementarity determining region sequences that exhibit convergent antigen binding.

In some embodiments, the methods disclosed herein are performed using a plurality of libraries. In some embodiments, analysis of data from multiple libraries allows identification of shared reactive antigens between libraries, e.g., antigens exhibiting TCR affinity that are present in multiple strains of a pathogen, multiple cancer types, multiple cancer patients, multiple autoimmune diseases, or multiple autoimmune conditions. In some embodiments, analysis of data from multiple libraries allows identification of distinct reactive antigens among libraries, e.g., antigens present in a subset of pathogen strains, cancers, conditions, or patients.

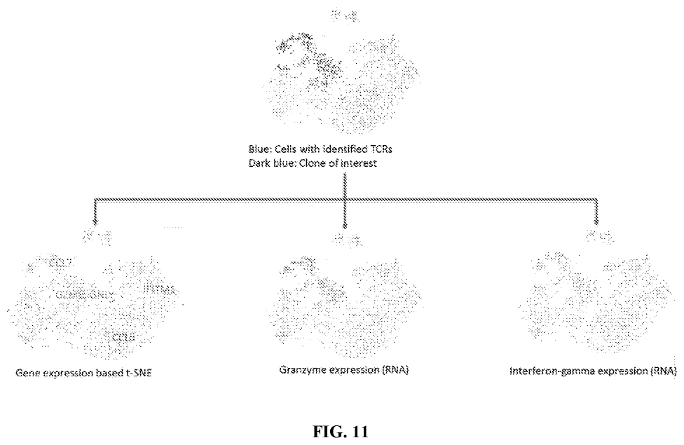

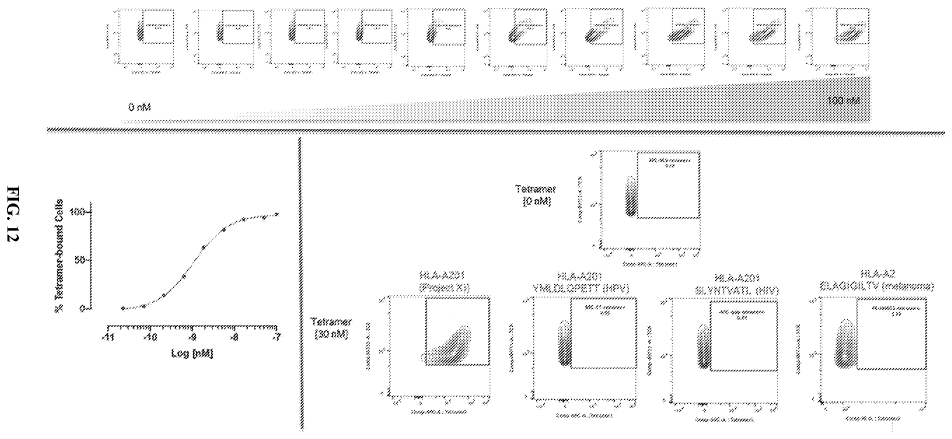

In some embodiments, cells interrogated with a peptide library of the disclosure are subjected to gene expression analysis (e.g., RNA-seq, qPCR). In some embodiments, gene expression analysis is conducted on cells identified as possessing a receptor exhibiting specificity for a peptide in a library of the disclosure (FIG. 11). For example, cells determined to express TCRs that bind to antigens in a pathogen library, cancer library, or autoimmune library are subjected to gene expression analysis. Gene expression analysis can be global or targeted. Genes analyzed for expression include, but are not limited to, genes with known functions, genes coding for immune effector molecules (e.g., perforin, granzyme, cytokines, chemokines), immune checkpoint molecules, pro-inflammatory molecules, anti-inflammatory molecules, lineage markers, integrins, selectins, lymphocyte memory markers, death receptors, caspases, cell cycle checkpoint molecules, enzymes, phosphatases, kinases, lipases, and metabolic genes. In some embodiments, gene expression analysis can be conducted concurrently with peptide library screening. In some embodiments, gene expression analysis can be conducted after analysis of peptide library screening results. In some embodiments, gene expression analysis can be conducted before analysis of peptide library screening results. In some embodiments, gene expression analysis allows for immunotyping of cells identified as of interest from peptide-receptor pairings produced using the methods described herein.

In some embodiments, a peptide library can be screened for a functional property. For example, an peptide library comprising a plurality of peptides, wherein the plurality of peptides comprise more than 10, more than 100, more than 500, more than 1,000, more than 2,000, more than 5,000, more than 10,000, more than 10⁶, more than 10⁷, more than 10⁸, more than 10⁹, or more than 10¹⁰ unique peptides, can be screened in a functional assay. In some embodiments, a peptide library is used for peptide library evaluation for the functional property or an additional functional property after an initial functional screen. For example, the peptide library can be contacted to a sample and then tested for induction of a functional property. A peptide library subset can be determined based on the peptide's ability to induce the functional property. The sample can be a biological sample. The sample can be cell sample. The sample can be a T cell sample. The sample can be from a subject. The subject can be a mammal. The subject can be a human.

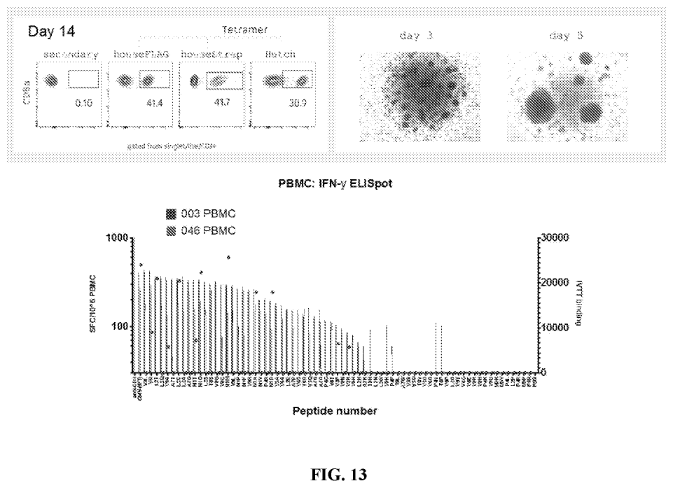

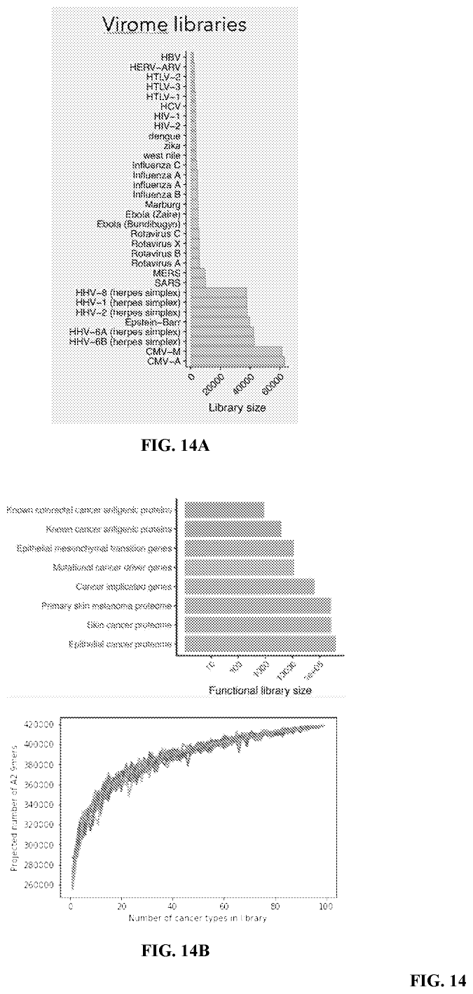

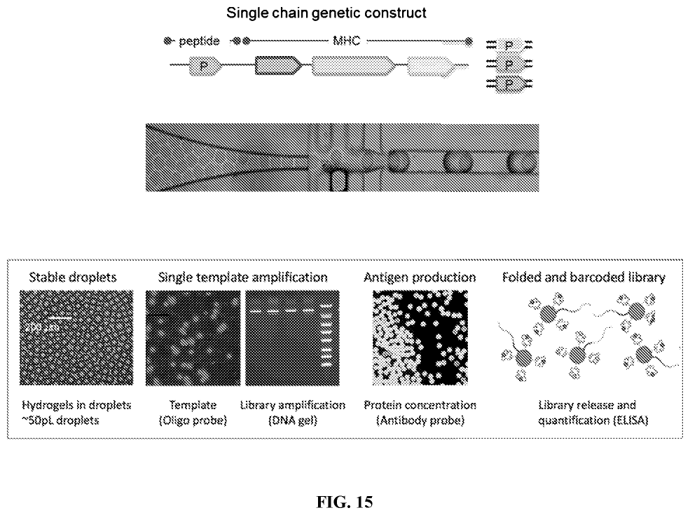

The methods and compositions described herein can be used for screening assays. For example, the peptide library can comprise a plurality of pMHC multimers as described herein that is contacted to T cell sample. In some embodiments, the peptide library can comprise a plurality of sc-pMHCs as described herein that is contacted to a T cell sample. After the contacting, proliferation of a T cell, cytotoxicity of a T cell, suppression of a T cell, suppression by a T cell, or cytokine production of a T cell can be determined for a pMHC multimer or sc-pMHC of the peptide library (FIG. 13). pMHC multimers or sc-pMHCs that can induce the functional property can then be made into a peptide library subset. For example, a peptide library subset can comprise pMHC multimers or sc-pMHCs that induce proliferation of a T cell upon binding to TCR, cytotoxicity upon binding to TCR, T cell suppression upon binding to TCR, suppression by a T cell upon binding to TCR, cytokine production upon binding to TCR, or any combination thereof. Proliferation can be determined by, for example, a dye-dilution assay (e.g., CFSE dilution assay), or quantification of DNA replication (e.g., BrdU incorporation assay). Cytotoxicity can be determined by, for example, assays that are based on release of an intracellular enzyme by dead cells (e.g., lactate dehydrogenase), dye exclusion assays (e.g., propidium iodide), or expression of cytolytic markers (e.g., granzyme, CD107a) by flow cytometry or qPCR. Cytokine production can be determined by, for example, ELISA, multiplex immunoassay, intracellular cytokine staining, ELISPOT, Western Blot, or qPCR. T cell suppression can be determined by, for example, co-incubating a T cell clone with effector cells and target antigen, and measuring proliferation, cytotoxicity, cytokine production, expression of activation markers, etc.

In some embodiments, the peptide libraries described herein can be used for isolating a receptor-peptide pair. In some embodiments, the method for isolating receptor-peptide pairs comprises contacting a plurality of receptors with a peptide library, wherein the peptide library has a diversity of more than 10, more than 100, more than 500, more than 1000, more than 2,000, more than 5,000, more than 10,000, more than 10⁶, more than 10⁷, more than 10⁸, more than 10⁹, or more than 10¹⁰ unique peptides; and generating a plurality of compartments, wherein a compartment of the plurality comprises a receptor of the plurality of receptors bound to a peptide of the peptide library, thereby isolating the receptor-peptide pair in the compartment. A receptor of the plurality of receptors can be, for example, a TCR, BCR, receptor tyrosine kinas (RTK), G-protein coupled receptor (GPCR), ligand-gated ion channel, cytokine receptor, chemokine receptor, or growth factor receptor, to name a few. In some embodiments, the receptor can be soluble. In some embodiments, the receptor can be bound to a surface. In some embodiments, the peptide library is an antigen library. The plurality of peptides can be a plurality of antigens. The plurality of peptides can be a plurality of pMHC multimers as described herein. The plurality of peptides can be a plurality of sc-pMHCs as described herein. The receptor-peptide pair can be a TCR-antigen pair. The receptor-peptide pair can be a BCR-antigen pair. In some embodiments, a peptide of the plurality of peptides comprises an identifier as described herein. A compartment can be a separate space, e.g., a well, a plate, a divided boundary, a phase shift, a vessel, a vesicle, a cell, etc.

The methods and compositions described herein can be used for identifying a receptor-peptide pair. In some embodiments, a method of identifying a receptor-peptide pair comprises contacting a plurality of receptors with a library of peptides wherein the library of peptides has a diversity of more than more than 10, more than 100, more than 500, 1000, more than 2,000, more than 5,000, more than 10,000, more than 10⁶, more than 10⁷, more than 10⁸, more than 10⁹, or more than 10¹⁰ unique peptides; compartmentalizing a receptor of the plurality of receptors bound to a peptide of the library of peptides in a single compartment, wherein the peptide comprises a unique peptide identifier; and determining the unique peptide identifier for each peptide bound to the compartmentalized receptor. A receptor of the plurality of receptors can be, for example, a TCR, BCR, receptor tyrosine kinas (RTK), G-protein coupled receptor (GPCR), ligand-gated ion channel, cytokine receptor, chemokine receptor, or growth factor receptor, to name a few. In some embodiments, the receptor can be soluble. In some embodiments, the receptor can be bound to a surface. In some embodiments, the peptide library is an antigen library. The plurality of peptides can be a plurality of antigens. The plurality of peptides can be a plurality of pMHC multimers as described herein. The plurality of peptides can be a plurality of sc-pMHCs as described herein. In some embodiments, a peptide of the plurality of peptides comprises an identifier as described herein. A receptor-peptide pair can be a TCR-antigen pair. A receptor-peptide pair can be a BCR-antigen pair.

The disclosure provides compositions and methods for the identification of pairings between, for example, peptides and cells, peptides and receptors, peptides and immune receptors, peptides and TCRs, peptides and BCRs, peptides and antibodies, ligands and cells, ligands and receptors, ligands and immune receptors, ligands and TCRs, ligands and BCRs, ligands and antibodies, agonists and cells, agonists and receptors, agonists and immune receptors, agonists and TCRs, agonists and BCRs, agonists and antibodies, antagonists and cells, antagonists and receptors, antagonists and immune receptors, antagonists and TCRs, antagonists and BCRs, antagonists and antibodies, antigens and cells, antigens and receptors, antigens and immune receptors, antigens and TCRs, antigens and BCRs, antigens and antibodies, epitopes and cells, epitopes and receptors, epitopes and immune receptors, epitopes and TCRs, epitopes and BCRs, and epitopes and antibodies. Pairings can be identified in different subjects (cross-sectional), in the same subject at different time points (longitudinally), or both. Identified peptides, receptors, or pairings can be associated with, for example, health, disease, early stage disease, mid stage disease, advanced disease, progressive disease, treatment response, remission, protective immunity, autoimmunity, etc. In some embodiments, a lack of peptide or lack of receptor specificity is associated with, for example, health, disease, early stage disease, mid stage disease, advanced disease, progressive disease, treatment response, remission, protective immunity, autoimmunity, etc.

In some embodiments, compositions and methods disclosed herein are used to identify antigen-specific T cell effector clones associated with protective immunity, non-protective immunity, or autoimmunity. In some embodiments, compositions and methods disclosed herein are used to identify antigen-specific T cell effector clones that exhibit anergy, exhaustion, tolerogenic properties, autoimmune properties, inflammatory properties, or anti-inflammatory properties (e.g., Tregs). In some embodiments, compositions and methods disclosed herein are used to identify antigen-specific T cell effector clones that exhibit certain effector or memory properties (e.g., naïve, terminal effector, effector memory, central memory, resident memory, T_{H}1, T_{H}2, T_{H}17, T_{H}9, T_{C}1, T_{C}2, T_{C}17, production of certain cytokines).

In some embodiments, a TCR, BCR, or antibody identified using compositions and methods disclosed herein are used as part of a therapeutic intervention. For example, a TCR sequence, TCR variable region sequence, or CDR sequence is transfected or transduced into T cells to generate T cells of the same specificity. The T cells can be expanded, polarized to a desired effector phenotype (e.g., T_{H}1, T_{C}1, Treg), and infused into a subject. In some embodiments, multiple TCR, BCR, or antibodies identified using compositions and methods disclosed herein are used in an oligoclonal therapy.

In some embodiments, a peptide, ligand, agonist, antagonist, antigen, or epitope identified using methods disclosed herein is used as part of a therapeutic intervention. In some embodiments, a peptide, antigen, or epitope is used to expand a population of cells ex vivo, e.g. using antigen presenting cells, artificial antigen presenting cells, immobilized peptide, or soluble peptide. In some embodiments, expanded cells are infused into a patient. In some embodiments, peripheral blood lymphocytes are expanded. In some embodiments, tumor-infiltrating lymphocytes (TILs) are expanded. In some embodiments, T_{H}1 cells are expanded. In some embodiments, cytotoxic T lymphocytes are expanded. In some embodiments, T regulatory cells are expanded.

In some embodiments, compositions and methods disclosed herein are used to identify antigens for use in development of a vaccine, e.g. a subunit vaccine, a vaccine eliciting coverage against a range of protective antigens, or a universal vaccine.

In some embodiments, compositions and methods disclosed herein can be used for diagnosis of a medical condition. In some embodiments, compositions and methods disclosed herein are used to guide clinical decision making, e.g., treatment selection, identification of prognostic factors, monitoring of treatment response or disease progression, or implementation of preventative measures.

Compositions and methods of the disclosure can comprise capture supports. In some embodiments, a peptide or nucleic acid of the disclosure is reversibly or irreversibly linked to a capture support. In some embodiments, a peptide or nucleic acid of the disclosure can be chemically linked to a capture support. In some embodiments, a peptide or nucleic acid of the disclosure can be covalently linked to a capture support. In some embodiments, a peptide or nucleic acid of the disclosure can be non-covalently linked to a capture support. In some embodiments, a peptide or nucleic acid of the disclosure can linked to a capture support by charged interactions, e.g., ionic bonds. In some embodiments, a peptide or nucleic acid of the disclosure can linked to a capture support by hydrogen bonds. In some embodiments, a peptide or nucleic acid of the disclosure can linked to a capture support by polar bonds. In some embodiments, a peptide or nucleic acid of the disclosure can be linked to a capture support by biotin-streptavidin or biotin-avidin interactions. In some embodiments, a peptide or nucleic acid of the disclosure can be conditionally released from a capture support, e.g., via chemical treatment or enzymatic processing.

In some embodiments, a capture support can be a solid surface. In some embodiments, a capture support can comprise a matrix. In some embodiments, a capture support can comprise a nanoparticle. In some embodiments, a capture support can comprise a bead. In some embodiments, a capture support can comprise a magnetic bead. In some embodiments, a capture support can comprise a hydrogel. In some embodiments, a capture support can be the inner surface of a water in oil emulsion droplet. In some embodiments, a capture support can comprise a nucleic acid molecule. In some embodiments, a capture support can comprise a protein. In some embodiments, a capture support can comprise an antibody or derivative thereof. In some embodiments, a capture support can comprise a gel. In some embodiments, a capture support can comprise a polymer. In some embodiments, a capture support can be charged. In some embodiments, a capture support can be fluorescent, e.g., labelled with a fluorescent dye or dyes.

In some embodiments, a peptide or nucleic acid of the disclosure can be cleaved from a capture support by enzymatic digestion. In some embodiments, a peptide or nucleic acid of the disclosure can be cleaved from a capture support by restriction enzyme digestion. In some embodiments, a peptide or nucleic acid of the disclosure can be cleaved from a capture support by chemical treatment or digestion.

### EXAMPLES

The following examples are included to further describe some aspects of the present disclosure, and should not be used to limit the scope of the invention.

### Example 1: Design of Unbiased 9-Mer Peptide

This Example demonstrates the identification of a 9-mer peptide library comprising the full chemical space for a peptide of a specific length.

This Example represented a departure from the past efforts where peptide libraries have been constrained by knowledge of targets of interest. These libraries were designed based on knowledge of physical interaction, target/partner characteristics, production limitations, or other parameters that have biased the breadth of the library. Thus, the libraries have been presented in a highly biased way. The library in this Example was designed to include all 9-mer peptides that were possible from the 20 amino acids encoded by the genetic code.

The library was designed to include all sequence combinations of 9-mers from the 20 known amino acids, e.g., 5×10 unique peptide sequences.

### Example 2: HLA-A2 Biased 9-Mer Peptide Chemical Space Interrogation

This Example demonstrates the identification of a 9-mer peptide library comprising the full chemical space for antigens of a specific length that were specific for interaction with major histocompatibility complex proteins, e.g., HLA-A2.

As in Example 1, this peptide library was not constrained by knowledge of targets of interest or interacting antigens. HLA-A2 has a well understood binding motif with key amino acids at positions 2 and 9 which can include I, V, or L. This library was designed to include all 9-mer peptides which have any of these sequences at both specified positions. The resulting library was a subset of the 9-mer library described in Example 1, as this library constrained 9-mer variability at positions 2 and 9.

The library was designed to include all sequence combinations of 9-mers from the 20 known amino acids with constraints at positions 2 and 9. A total of 1×10 \vec{ }10 unique 9-mer peptide sequences were identified as HLA-A2 restricted. Similar restrictions are analogous for other MHC complexes.

### Example 3: Human Virome 9-Mer Peptide Library

This Example demonstrates the identification of a 9-mer peptide library comprising the full chemical space for human viral antigens of this specific length.

Viruses selected for inclusion in the peptide library were curated human hosted viral proteomes, as indicated by the online database Uniprot, and supplemented with taxa identifiers for strains identified by programmatic search of Uniprot Proteomes. Full and partial proteomes were downloaded by proteome identifier from Uniprot using the REST API. Proteome coverage of selected strains was verified manually. The library was then further expanded to include all proteomes from available Orthohantaviruses, as well as additional diversity around highly variable taxa (HIV and Influenza).

Every 9-mer from each protein in the identified proteomes were included in this library. This library was also a subset of the 9-mer unbiased library described in Example 1, as this library constrained 9-mer variability to be derived from the curated human viral proteome.

An additional library was identified as 9-mers restricted to one of the MHC complex proteins, such as described in Example 2. A total of 1.5×10\hat{ }5 unique 9-mer peptides of the 3×10\hat{ }6 peptides were identified as HLA-A2 restricted.

### Example 4: Cytomegalovirus 9-Mer Peptide Library

This Example demonstrates the identification of a 9-mer peptide library covering the complete proteome of Cytomegalovirus (CMV).

This library was designed to include every 9-mer from each protein of the CMV proteome. The resulting library included 7×10∝unique 9-mer peptides. This library was also a subset of the 9-mer human viral library described in Example 3, as this library constrained 9-mer variability to be derived from CMV.

An additional library was identified as 9-mers restricted to one of the MHC complex proteins, such as described in Example 2. A total of 4×10\hat{ }3 unique 9-mer peptides of the 7×10∝peptides were identified as HLA-A2 restricted.

### Example 5: Cytomegalovirus pp65 Protein 9-Mer Peptide Library

This Example demonstrates the identification of a 9-mer peptide library comprising the complete protein sequence of Cytomegalovirus (CMV) protein, pp65.

Every 9-mer from the pp65 protein was included in this library. The resulting library included 571 unique 9-mer peptides. This was also a subset of the 9-mer library described in Example 4, as these 9-mers were associated with the pp65 protein.

An additional library was identified as 9-mers restricted to one of the MHC complex proteins, such as described in Example 2. A total of 26 unique 9-mer peptides of the 571 peptides were identified as HLA-A2 restricted.

### Example 6: Epitope Specific Positional Scan Mutation for 9-Mer Peptide Library

This Example demonstrates the identification of a 9-mer peptide library comprising the complete mutational scan of an epitope.

A library of 9-mers with single mutations, as described by Hoppes, et al. in J Immunol, 2014, 193, was designed for the NLVPMVATV epitope of pp65 protein. The resulting library included 172 unique 9-mer peptides. This was also a subset of the 9-mer library described in Example 1, as these 9-mers comprised positional mutations throughout the NLVPMVATV-based 9-mer.

A library of 9-mers with two mutations was designed for the NLVPMVATV epitope of pp65 protein. The resulting library included 13,168 unique 9-mer peptides.

A library of 9-mers with three mutations was designed for the NLVPMVATV epitope of pp65 protein. The resulting library included 589,324 unique 9-mer peptides.

A library of 9-mers with all mutations was designed for the NLVPMVATV epitope of pp65 protein. The resulting library included 5.12×10¹¹ unique 9-mer peptides.

### Example 7: Production of 9-Mer Peptide Library

The peptide libraries described in any of the previous Examples are generated according to methods known in the art, or synthetically produced by a commercial vendor or using a peptide synthesizer according to manufacturer's instructions.

### Example 8: In Vitro Translation of Peptide MHC Library

This Example demonstrates cell-free synthesis (CFPS) of a protein.

Cell-free protein synthesis (CFPS) of a peptide library enables the production of a broad range of various peptides. Obtaining a high yield by CFPS requires the usage of bacterial systems, in which the first amino acid of the translated sequence is N-formylmethionine (fMet). This residue differs from methionine by containing a neutral formyl group (HCO) instead of a positively charged amino-terminus (NH₃⁺). Therefore, each peptide library variant will contain fMet. However, the architecture of the peptide binding groove of MHC class I molecules is designed to specifically accommodate only positively charged amino-terminus of any given peptide and will fail to adequately fit a peptide that has a sequence initiation with fMet. An unsuccessful peptide loading will influence folding and will result in a misfolded, nonfunctional MHC, as both processes are linked. Although, bacteria can utilize endogenous aminopeptidases to cleave the fMet, its removal could be either incomplete or abolished, depending on the identity of the second amino acid in the sequence. For example, methionine aminopeptidase excises inefficiently between fMet and asparagine. Consequently, a CMV derived peptide, a model peptide in this system, will be ultimately produced as fMet-NLVPMVATV in a single-chain design; the entire molecule will not fold correctly and will not bind its cognate T-cell receptor. This result is anticipated if the protein will be produced in bacterial CFPS system that is made from a crude cell extract. Moreover, in a library context, a single individual template could result in peptides with the fMet, or without it, or a mixture of both if the processing is merely inefficient. In reconstituted CFPS systems that are composed of only purified components and completely lack methionine aminopeptidases, all library variants will start with an fMet residue.

To solve this problem, constructs were engineered to include genes encoding an enzymatic cleavage domain and a library polypeptide. Removal of at least the initial methionine amino acid allowed successful peptide folding and loading onto MHC protein. Furthermore, removal of the initial methionine amino acid provided a greater upper limit of peptide library diversity, e.g., 20×, where x is the length of the peptide, while inclusion of this residue will restrict the library diversity to 20^{(x-1)}.

In this Example, peptides were synthesized under cell-free conditions. All CFPS components were thawed and mixed on ice and then moved to the relevant temperature to initiate the reaction. Reagents were added: 40% (V/V) PURExpress solution A, 30% (V/V) of PURExpress solution B (E6800L, New England Biolabs, Inc.), 0.8 U/μl reaction of RNase inhibitor (10777019, ThermoFischer Scientific), 4% (V/V) of each disulfide enhancers 1 and 2 (E6820L, New England Biolabs, Inc.), 0.004 U/μl reaction of protease diluted in PBS (Invitrogen), nuclease-free water and 20 ng/μl reaction of the corresponding plasmid DNA encoding the desired CFPS product. Four different temperatures of CFPS were tested: 20, 25, 30 and 37° C. In each indicated time point, samples were taken and the reactions were stopped by placing the tubes on ice and adding EDTA to a final concentration of 2 mM.

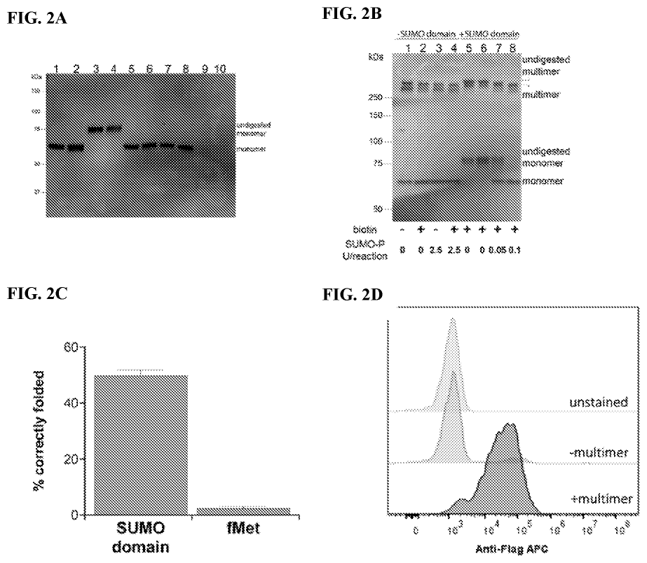

FIG. 2A demonstrates enzymatic cleavage to increase peptide diversity. Lane pairs 1-2, 3-4, 5-6, 7-8 and 9-10 represent CFPS reactions performed on a template without a cleavage moiety, a template with a cleavage moiety where no protease was added, a template with a cleavage moiety in which the protease was added after the reaction was completed, a template with a cleavage moiety in which the protease was present during the reaction, and a reaction that lacked template, respectively. Samples in odd numbered lanes were prepared for gel electrophoresis under reduced conditions with the addition of 100 mM DTT. All reactions were terminated by placing the tubes on ice after 4 hr at room temperature. 4 U/reaction of protease were added to samples 3-8. In the reactions loaded in lanes 5-6, the protease was added after the tube was placed on ice together with 10 mM EDTA and then transferred to room temperature for additional 3.5 hours before placing on ice again.

Western blotting was performed to determine total protein yield. Each CFPS sample was mixed with water, 4× sample buffer and 1M DTT, boiled at 95° C. for 5 minutes, and then loaded on a 10% SDS-PAGE gel. Samples were blotted using an HRP-anti-FLAG antibody.

FIG. 2B show samples from the CFPS reaction that contained multimer and monomer templates with or without the cleavage moiety. The samples were blotted and detected with an anti-FLAG HRP antibody. Reactions were terminated by placing the tubes on ice after 4 hr at room temperature.

### Example 9: Assessing 3-D Structure of In Vitro Translated Protein

This Example demonstrates the CFPS protein folded into a recognizable 3-dimensional structure.

In this Example, CFPS protein generated in Example 8 was tested for conformational recognition by an antibody. Proteins that are misfolded or unfolded are not recognized by the antibody. The following Example demonstrates that the CFPS protein was folded and conformationally recognized by the antibody following cleavage of the enzymatic cleavage domain.

Protein expression was measured through an ELISA. Plates were coated with anti-streptavidin antibody (410501, Biolegend) diluted in 100 mM bicarbonate/carbonate coating buffer and incubated over-night at 4° C. Then the plates were washed three times by filling the wells with washing buffer (PBS supplemented with 0.05% tween-20) and blocked for 2 hr at room temperature by filling the wells with blocking buffer (washing buffer supplemented with 2% (V/V) BSA). The wells were then filled with serial dilutions of each CFPS protein in blocking buffer followed by 1 hr incubation at room temperature. Then the plates were washed three times with washing buffer and incubated with 0.15 μg/ml horseradish peroxidase-conjugated antibodies specific to the protein diluted in blocking buffer for 1 hr at room temperature.

After three more washes, colors were developed with the addition of 3,3′,5,5′ tetramethylbenzidine substrate to each well and the reaction was stopped by adding a commercial stop solution. The absorbance at 450 nm was measured using a plate reader. Values are averages of duplicates. Plates were covered with adhesive plastic and were gently agitated on a rotator during all incubations. The concentration of each sample was interpolated from a standard curve of a positive control protein.

FIG. 2C demonstrates that peptides generated by CFPS and subjected to proteolytic cleavage fold into a recognizable 3-dimensional structure. ELISA was used to detect linear epitopes or a conformation epitope and the correctly folded percentage was calculated. The protease was added to both CFPS reactions. The figure indicates whether protease-cleaved peptide or uncleaved (Met-containing) peptide demonstrated correct folding by recognition with the conformational epitope antibody.

FIG. 2D provides binding of a single-chain peptide-MHC (sc-pMHC) multimer to antigen-specific T cells. Multimers were produced by CFPS and enzyme cleavage. T cells were incubated with multimers, then stained with a fluorescent detection antibody and analyzed by flow cytometry. For FACS staining, CMV enriched T cells (Donor 153, Astarte 3835FE18, Cat #1049) were used. Wells of a 96-well round bottomed microtiter plate were filled with T cells and the cells were washed once with ice-cold FACS buffer (D-PBS, 2 mM EDTA and 2% (V/V) fetal bovine serum), spun at 300 g at 4° C., and the supernatant was removed. Then the relevant wells were blocked with Fc receptor blocking solution for 30 minutes at 4° C. under gentle agitation, washed with FACS buffer, and the supernatant was removed. FACS buffer was added to the compensation control wells.

In the next step, the cells were incubated for 30 minutes at 4° C. with either 20 nM positive control or dilutions of samples taken from the indicated CFPS reactions in Example 8 and then washed once with FACS buffer. 100 nM detection antibody diluted in FACS buffer was added to each well and the plate was incubated at 4° C. for 30 minutes in the dark, followed by two washes with PBS, and staining with fixable viability dye APC-efluor780 (1:8000 dilutions, 50 μl/well) for 15 min at room temperature. The plate was then washed twice with FACS buffer and fixed with fixation buffer PBS, 3.7% formaldehyde (V/V), 2% FBS (V/V)). Finally, the samples were transferred to FACS tubes for analysis.

### Example 10: Production of Peptide Library in Mammalian Cells

Peptides were produced either by cell-free protein synthesis, as described in Example 8, in mammalian cells, or synthetically as in Example 7.

For mammalian expression, a construct encoding the CMV peptide was designed with a C-terminal Flag-tag with and without a C-terminal His-tag in a mammalian expression vector. Peptides were expressed by transient transfection in Expi293F or ExpiCHO-S cells (Life Technologies) according to the manufacturer's recommendations.

Peptides were purified from cell culture supernatants with anti-Flag affinity chromatography (Genscript) or by Ni-affinity chromatography. Size exclusion chromatography (SEC) was performed on a hydrophilic resin (GE Life Sciences) pre-equilibrated in 20 mM HEPES, 150 mM NaCl, pH 7.2.

Alternatively, peptides were purified by Ni-affinity chromatography without SEC purification, using a column buffer of 23 mM sodium phosphate, 500 mM sodium chloride, 500 mM imidazole, pH 7.4.

Peptides produced in mammalian cells were quantitated by UV at 280 nm, whereas CFPS-produced peptides were quantitated by a sandwich ELISA relative to a standard protein.

### Example 11: Attaching Peptide Identifier to a Library Peptide

This Example demonstrates labeling a library peptide with a peptide identifier.

CMV peptide was produced either synthetically as in Example 7, by cell-free protein synthesis, as described in Example 8, or in mammalian cells as described in Example 10.

Peptides produced as described herein were labelled with one or more peptide identifier (e.g., DNA fragments). Each peptide identifier was either commercially synthesized (Integrated DNA Technologies) or PCR amplified. Labelling was achieved by mixing 50% v/v peptide and 50% v/v peptide identifier and confirmed by upward shift on western blot.

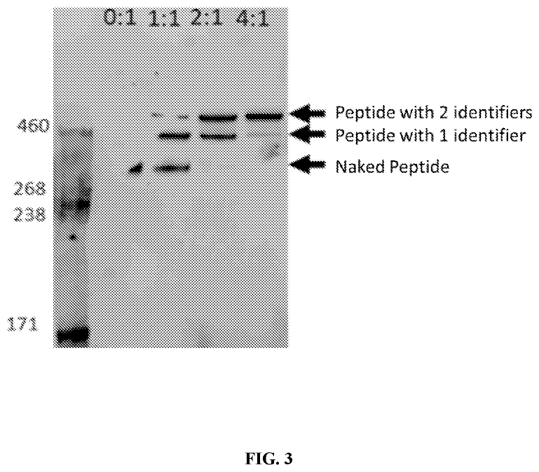

FIG. 3 shows the western blot of the CMV peptide with one or more peptide identifiers. The bottom arrow indicates naked peptide. The middle arrow indicates peptide with one peptide identifier. The upper arrow indicates peptide with two peptide identifiers.

### Example 12: HLA-A2 9-Mer Peptide Library

This Example demonstrates the ability to generate a 9-mer peptide library comprising the full chemical space for a length of antigen specific for HLA-A2.

This is a departure from the past efforts where specific targets of interest have been identified via physical interaction and then presented in a highly biased way. HLA-A2 has a well understood binding motif with key amino acids at positions 2 and 9 which can include I, V, or L. The library in this Example is designed to only include all 9-mer peptides which have these sequences at both specified positions, resulting in 1×10\hat{ }10 peptides.

Constructs are engineered to include genes encoding for an enzymatic cleavage domain and one of the 1×10\hat{ }10 unique 9-mer peptides.

The peptide library is generated according to similar methods as described in Example 8. The peptide library is then loaded onto HLA-A2 molecules to generate peptide/MHC (pMHC) library.

The resulting pMHC library may be used in T cell screens to determine antigen-reactive T cells. See for example, Simon et al, Cancer Immunol Res, 2014, 2(12):1230-1244.

### Example 13: Peptide Library Binding to Cells

This Example demonstrates detection of peptide binding to cells.

To test functionality of the peptides labeled with peptide identifiers, peptide-specific (CMV) and non-peptide (HPV) specific T-cells were obtained (Astarte Biologics). Frozen T-cells were thawed according to manufacturer's guidelines. Cells were blocked with 20% v/v Fc block and 0.1 mg/ml salmon sperm for 30 mins at 4° C. Cells were then incubated with 10% V/V peptide identifier labeled peptides in FACS buffer (D-PBS, 2 mM EDTA and 2% (V/V) FBS) for 30 mins at 4° C. and washed.

Cells were further divided into two fractions, where peptide binding was detected using flow cytometry and identifiers were detected by qPCR. For protein-based detection, cells were incubated with anti-Flag antibody (Biolegend) 2% v/v for 30 mins at 4° C. and washed. Finally, cells were fixed in fixation buffer (D-PBS, 3.7% formaldehyde and 2% FBS) and analyzed on a flow cytometer.

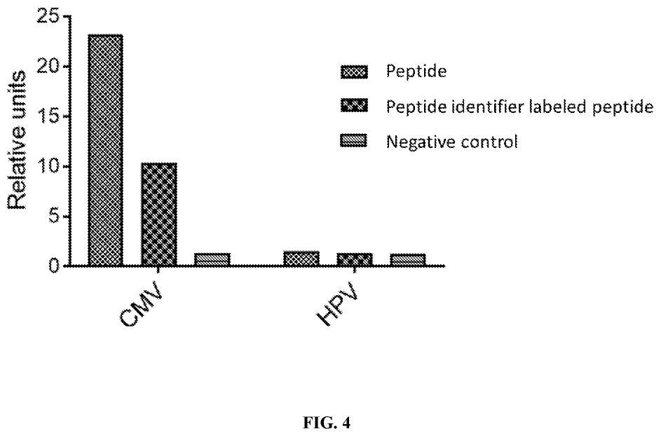

FIG. 4 shows relative binding of naked peptide, peptide labeled with peptide identifier, or negative control to peptide specific or non-peptide specific T cells. Naked peptide and peptide labeled with peptide identifier showed binding to peptide specific T cells as compared to non-peptide specific T cells.

For peptide identifier-based detection, cells were lysed using the cell lysis and RNA stabilization kit (Life Technologies Corporation) and a qPCR master mix was prepared according to the manufacturer's protocol. Ct values were normalized to an internal control using primers specific for a housekeeping gene (e.g., Rpl13). The relative values were compared to those derived from T cells without peptide using the delta-delta Ct method.

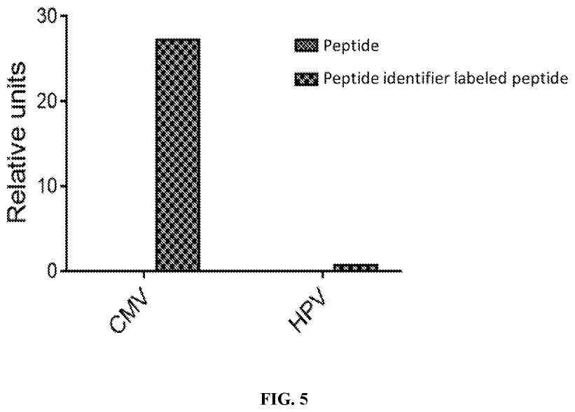

FIG. 5 shows that relative amount of peptide identifier labeled peptide normalized to a housekeeping gene. Peptide specific T cells incubated with peptide identifier labeled peptide had vastly more signal than non-peptide specific T cells incubated with peptide identifier labeled peptide, indicating a specific interaction between the T cells and the peptide. In addition, naked peptide had little to no detectable signal for both peptide specific and non-peptide specific T cells.

### Example 14: Identification of TCR Antigen Specificity Profiles

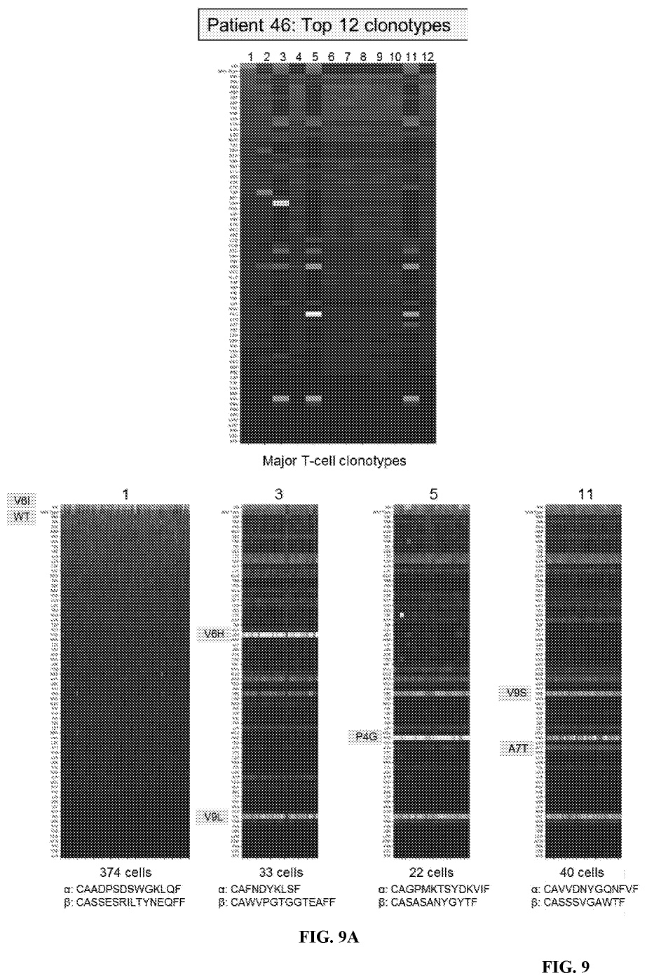

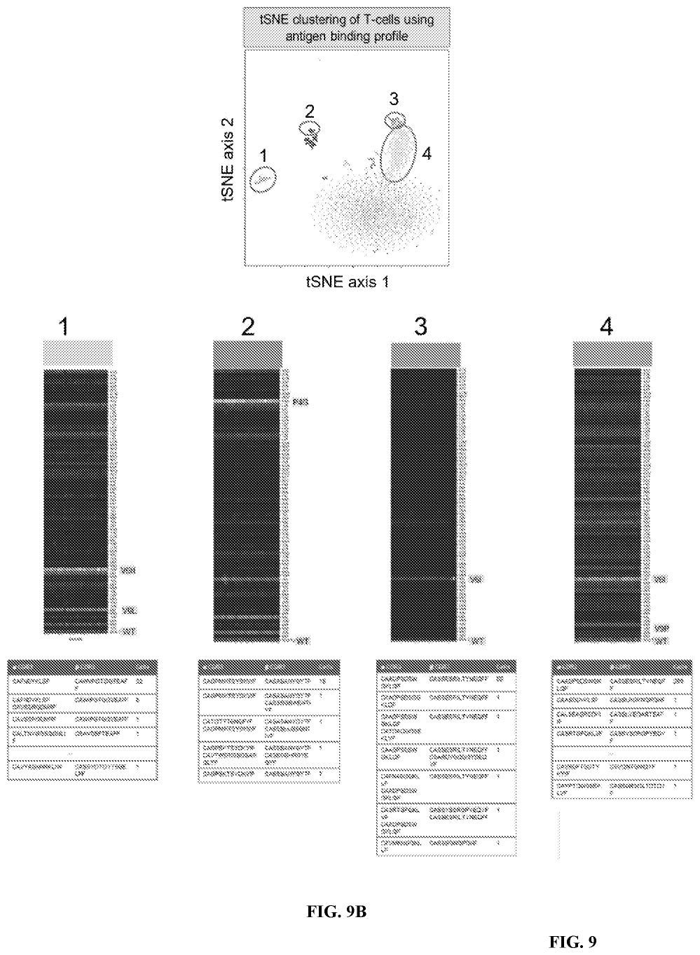

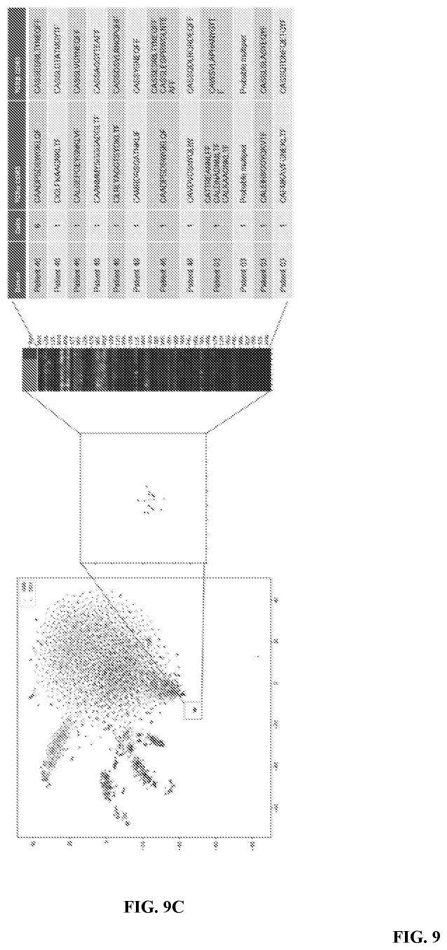

A 9-mer library comprising a complete mutational scan of the NLVPMVATV epitope is designed as described in Example 6. sc-pMHC comprising the 9-mers are synthesized as described in Example 8, and identifiers attached as described in Example 11. The library is incubated with a plurality of T cells, and T cells with are sorted into single-cell compartments. T cells are lysed, and nucleic acids from the lysed T cells comprising identifiers are produced. These nucleic acids are pooled and sequenced. Identifiers in reads allow matching of peptide identifiers to T cell sequences from the same compartment. TCR-antigen specificity profiles are determined by identifying a TCR sequence (e.g., variable region, hypervariable region, or CDR) from a compartment, and quantifying peptide identifier reads from the same compartment (FIG. 9A). Epitope mutations in an antigen of an identified TCR-antigen pair are identified that result in increased or decreased TCR binding affinity.

### Example 15: Identification of TCRs that Bind Target Antigens

Sequencing data is generated as described in Example 14. For each peptide identifier sequenced, corresponding TCR sequences are identified (e.g., variable region, hypervariable region, or CDR). Multiple TCRs are identified that exhibit binding affinity for peptides of the peptide library, and multiple peptides are identified that exhibit binding affinity for specific TCRs (FIG. 9B).

### Example 16: Convergence of Diverse TCRs

Experiments are conducted using the methods described in Example 14 and Example 15. T cells are primary T cells derived from different subjects. TCRs from different individuals are identified that exhibit binding affinity for peptides of the peptide library (FIG. 9C).

### Example 17: CMV Antigen Discovery and Vaccine Design

This Example demonstrates use of compositions and methods disclosed herein for discovery of specific antigens and T cell receptor sequences, and subsequent design of vaccines and cell therapies.

Subjects scheduled to undergo hematopoietic stem cell transplantation (HSCT) are enrolled in the study. Blood is drawn on day 0, and day 30 post-HSCT. T cells are isolated from the blood and cultured. The cultured T cells are incubated with a sc-pMHC library of the disclosure (e.g., a library comprising peptides derived from CMV genomes, transcriptomes, or proteomes with positional scanning), and cells are sorted into single cell compartments.

T cells are lysed, and nucleic acids from the lysed T cells comprising identifiers are produced. These nucleic acids are pooled and sequenced. Identifiers in reads allow matching of peptide identifiers to T cell sequences from the same compartment. TCR antigen specificity profiles are determined by identifying a TCR sequence (e.g., variable region, hypervariable region, or CDR) from a compartment, and quantifying peptide identifier reads from the same compartment. For each peptide identifier sequenced, corresponding TCR sequences are identified. Multiple TCRs are identified that exhibit binding affinity for one or more peptides of the peptide library, and multiple peptides are identified that exhibit binding affinity for one or more TCRs. Subjects are classified as CMV seropositive or seronegative, and are additionally classified based on CMV control or reactivation. Results from subjects are compared. Peptides and TCR sequences are identified that are associated with control of CMV, and are used to design CMV vaccines and cell therapies.

### Example 18: Vaccine and TCR Cell Therapies for Checkpoint Inhibitor Non-Responders

This Example demonstrates use of compositions and methods disclosed herein for discovery of specific antigens and TCR sequences associated with response to checkpoint inhibitor therapy, and subsequent design of vaccines and cell therapies.

Subjects scheduled to undergo checkpoint inhibitor therapy for non-small cell lung cancer (NSCLC) or colorectal cancer (CRC) are enrolled in the study. Longitudinal biopsies are obtained from subjects before the administration of the checkpoint inhibitor, and after the checkpoint inhibitor is administered and time for a therapeutic effect is allowed. T cells are isolated from the biopsies and cultured. The cultured T cells are incubated with a sc-pMHC library of the disclosure (e.g., a library comprising peptides derived from NSCLC/CRC genomes, transcriptomes, or proteomes), and cells are sorted into single cell compartments.

T cells are lysed, and nucleic acids from the lysed T cells comprising identifiers are produced. These nucleic acids are pooled and sequenced. Identifiers in reads allow matching of peptide identifiers to T cell sequences from the same compartment. TCR antigen specificity profiles are determined by identifying a TCR sequence (e.g., variable region, hypervariable region, or CDR) from a compartment, and quantifying peptide identifier reads from the same compartment. For each peptide identifier sequenced, corresponding TCR sequences are identified. Multiple TCRs are identified that exhibit binding affinity for one or more peptides of the peptide library, and multiple peptides are identified that exhibit binding affinity for one or more TCRs.

Subjects can be followed longitudinally, and the assays performed on biopsies during two or more cycles of treatment with checkpoint inhibitor.

Subjects are classified as checkpoint inhibitor responders or non-responders. Results from subjects are compared. Peptides and TCR sequences are identified that are associated with successful response to checkpoint inhibitor therapy. Identified peptides and TCR sequences can be confirmed in second or subsequent cycle of treatment with checkpoint inhibitor, or in subsequently enrolled subjects. Identified peptides and TCR sequences are used to design cancer vaccines and cell therapies.

### Example 19: Universal Influenza Vaccine

This Example demonstrates use of compositions and methods disclosed herein for discovery of specific antigens and TCR sequences associated with immune responses to influenza strains, and subsequent design of vaccines, including a universal influenza vaccine.

Subjects are enrolled to be vaccinated or infected with various flu strains. Subjects are infected with influenza, vaccinated with a live attenuated influenza strain, or vaccinated with an influenza subunit vaccine.

Longitudinal blood samples are obtained from subjects on day −7 (before infection/vaccination), day 10 post-infection/vaccination, and day 45 post-infection/vaccination.

T cells are isolated from the blood samples and cultured. The cultured T cells are incubated with a sc-pMHC library of the disclosure (e.g., a library comprising peptides derived from influenza genomes, transcriptomes, or proteomes with positional scanning), and cells are sorted into single cell compartments.

T cells are lysed, and nucleic acids from the lysed T cells comprising identifiers are produced. These nucleic acids are pooled and sequenced. Identifiers in reads allow matching of peptide identifiers to T cell sequences from the same compartment. TCR antigen specificity profiles are determined by identifying a TCR sequence (e.g., variable region, hypervariable region, or CDR) from a compartment, and quantifying peptide identifier reads from the same compartment. For each peptide identifier sequenced, corresponding TCR sequences are identified. Multiple TCRs are identified that exhibit binding affinity for one or more peptides of the peptide library, and multiple peptides are identified that exhibit binding affinity for one or more TCRs. Analysis of peptide-MIIC convergence from different subjects subjected to infection/vaccination allows identification of protective antigens. Protective antigens are concatenated into a vaccine to provide broad or universal protection from multiple strains of influenza.

### Example 20: Treg Therapy for Diabetes

This Example demonstrates use of compositions and methods disclosed herein for discovery of specific antigens and TCR sequences associated with autoimmunity, and subsequent design of a tolerogenic cell therapy.

One arm of the study utilizes post-mortem tissue samples collected from subjects with stage 0/1 type 1 diabetes (and matched healthy controls). Tissue samples include beta islands, blood, spleen, lymph node, and bone marrow. In a second arm of the study, living subjects are enrolled with stage 0/1 type 1 diabetes (and matched healthy controls). Blood samples are drawn from the subjects periodically.

T cells are isolated from the blood and tissue samples and cultured. The cultured T cells are incubated with a sc-pMHC library of the disclosure (e.g., a library comprising peptides derived from genomes, transcriptomes, or proteomes of healthy or autoimmune human subjects), and cells are sorted into single cell compartments.

T cells are lysed, and nucleic acids from the lysed T cells comprising identifiers are produced. These nucleic acids are pooled and sequenced. Identifiers in reads allow matching of peptide identifiers to T cell sequences from the same compartment. TCR antigen specificity profiles are determined by identifying a TCR sequence (e.g., variable region, hypervariable region, or CDR) from a compartment, and quantifying peptide identifier reads from the same compartment. For each peptide identifier sequenced, corresponding TCR sequences are identified. Multiple TCRs are identified that exhibit binding affinity for one or more peptides of the peptide library, and multiple peptides are identified that exhibit binding affinity for one or more TCRs.

Results from subjects are compared. Peptides and TCR sequences are identified that are associated with type 1 diabetes. Identified peptides and TCR sequences are used to tolerogenic cell therapies, e.g., ex vivo expanded oligoclonal Treg-polarized T cells expressing TCR specific for autoimmune antigens.

### Example 21: Production of a Porous Hydrogel

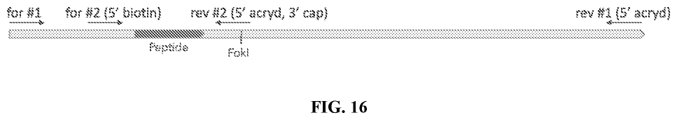

This Example demonstrates production of porous hydrogels that can be used in compositions and methods of the disclosure. Hydrogel beads were produced by mixing acrylamide monomer units and bis-acrylamide crosslinker units at a variety of relative concentrations along with a mixture of acrydated oligonucleotide primers, encapsulating in droplets using a microfluidic drop-maker, and incubating the mixture until crosslinking was complete. In this Example, the pre-crosslinked aqueous mix included 0.75% bis-acrylamide, 3% acrylamide, 5 μM 5′-acrydated reverse primer #1, 25 μM 3′-capped (phosphorylated) and 5′-acrydated reverse primer #2 (FIG. 16), 0.5% ammonium persulfate, in 10% TEBST (Tris-EDTA-buffered saline plus Tween-20). Primers can be designed to include sequences for enzymatic cleavage, for example sequences targeted by a restriction enzyme, to allow liberation of part of the primer from the hydrogel. Any suitable restriction enzyme can be used. In this example, reverse primer 1 included an XhoI digestion site and reverse primer 2 included a FokI digestion site. All reagents of the aqueous mixture were combined and stirred. The mixture was supplemented with 1.5% TEMED and 1% of 008-FluoroSurfactant, encapsulated in droplets, incubated at room temperature for 1 hr, and then transferred into an oven at 60° C. for overnight incubation, thus forming the hydrogels. The hydrogel beads were washed once with 20% 1H,1H,2H,2H-perfluoro-1-octanol (PFO), then washed three times with TEBST, and then washed three times with low TE (1 mM Tris-Cl pH 7.5, 0.1 mM EDTA). Hydrogel beads were stored in TEBST at 4° C. until use.

### Example 22: PCR of Full-Length Antigen-Encoding Templates onto Hydrogels (PCR1)

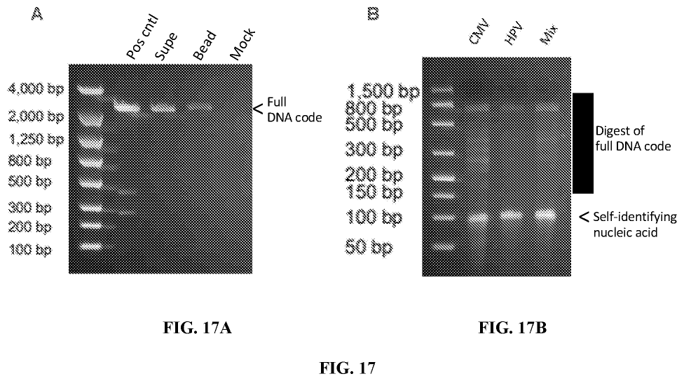

This Example demonstrates PCR of full length antigen-encoding templates onto hydrogels. Linear DNA templates encoding single chain multimeric peptide-MHC were PCR-amplified onto hydrogel beads in drops under single template conditions, where each drop gets at most a single DNA template. 1.4 mL hydrogel beads produced in Example 21 were mixed together with PCR components as follows in a 2 mL reaction volume: 400 μL Q5 reaction buffer (New England Biolabs), 40 μL 10 mM dNTP, 40 μL 25 μM forward primer #1, 40 μL 1 μM of non-acrydated reverse primer #1 (FIG. 16), 40 μL 0.1 μg/ul linear DNA template (or mix of templates), 8 μL 20% IGEPAL, and 20 μL Q5 DNA polymerase (New England Biolabs). The mixture was encapsulated in drops and subjected to 35 cycles of PCR. After drop lysis by addition of an equal volume of 100% perfluorooctanol (PFO), hydrogels were washed with 10 volumes of low TE five times. Aliquots (10 μL ea) of hydrogel beads were digested with a restriction enzyme that cuts within reverse primer #1 (in this example, XhoI) for 1 h at 37° C., and run on a 1.2% agarose gel to quantify yield and quality of amplicons on hydrogels. As shown in FIG. 17A, full length antigen-encoding templates were PCR amplified onto hydrogels (“bead”).

### Example 23: PCR of an Identifier (PCR2)

This Example demonstrates PCR amplification of identifiers on hydrogels generated in examples 21 and 22. Any suitable identifier disclosed herein can be used. In this example, a self-identifier was used that corresponds to all or a part of a nucleic acid sequence that encodes the peptide that it identifies. Washed hydrogel beads after PCR1 were digested with shrimp alkaline phosphatase (New England Biolabs) to remove the 3′ cap on reverse primer #2 and then further washed 5 times with 10 volumes of low TE. 300 μL hydrogel beads were mixed together with PCR components as follows in a 400 μL reaction volume: 80 μL Q5 reaction buffer (New England Biolabs), 8 μL 10 mM dNTP, 8 μL 25 uM 5′-biotinylated forward primer #2, 1.6 μL 20% IGEPAL, and 4 μL Q5 DNA polymerase (New England Biolabs). The mixture was encapsulated in drops and subjected to 20 cycles of PCR. After drop lysis by addition of an equal volume of 100% PFO, hydrogel beads were washed five times with 10 volumes of low TE. Small aliquots of hydrogel beads were digested with a restriction enzyme that cuts within reverse primer 2 (in this example, FokI) for 1 h at 37° C. and run on a 1.2% agarose gel to quantify the yield and quality of identifier amplicons on hydrogels. As shown in FIG. 17B, identifiers were PCR amplified onto hydrogens (“self-identifying nucleic acid”). Three separate bead preps were analyzed: one with a template corresponding to a CMV peptide, one with an HPV peptide, and one with a mixture of templates encoding both peptides (mix). The self-identifying nucleic acid fragment produced by PCR2 is indicated at −100 bp.

### Example 24: In Vitro Transcription/Translation (IVTT) of Single Chain Multimeric Peptide-MHC

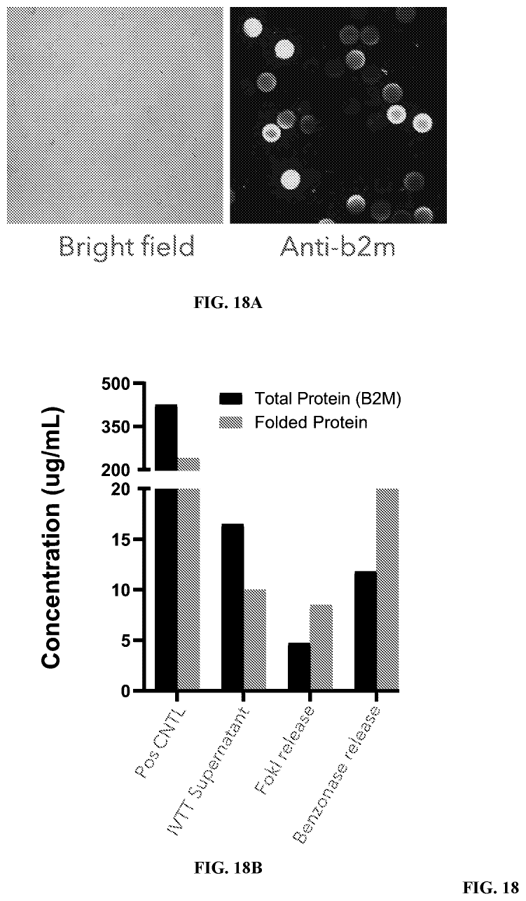

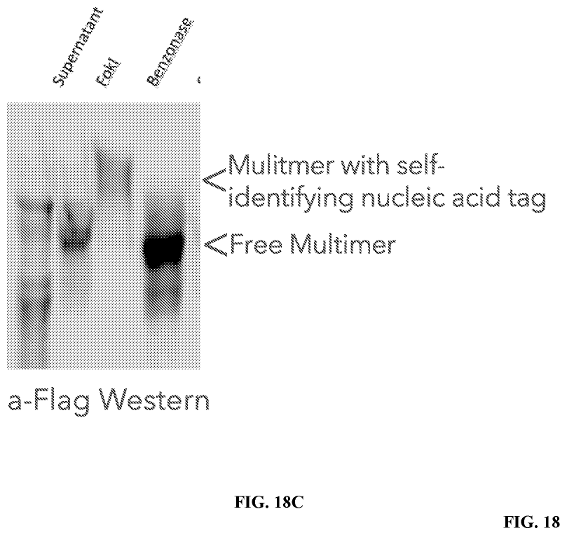

This Example demonstrates single chain peptide-MHC can be in vitro transcribed and translated, for example, using antigen-encoding DNA templates on hydrogels as generated in examples 21 and 22. 120 μL of hydrogel beads were co-encapsulated in drops with 240 μL of IVTT master mix, including 120 μL PURExpress solution A (New England Biolabs), 90 μL PURExpress solution B (NEB), 6 μL RNAse OUT (Invitrogen), 12 μL each Disulfide Bond Enhancer #1 and #2 (NEB), and 12 μL Ulp1 protease (Invitrogen). Drops were incubated at 22° C. for 20 hours, without shaking. D-Biotin was added to the IVTT reactions to a final concentration of 500 μM prior to breaking drops by addition of an equal volume of 100% PFO. Hydrogel beads were washed five times with 10 volumes of PBS plus 2% BSA. An aliquot of hydrogels was subjected to immunofluorescent staining with 1:10 dilution of Alexa-488-labeled anti-beta-2-microglobulin (B2M) antibody (R&D Systems) in PBS plus 2% BSA for 1 hour at room temperature, followed by five 10-fold washes in PBS plus 2% BSA, and imaging by confocal microscopy (Imagexpress Micro, Molecular Devices, FIG. 18A). Staining was observed in 21% of beads, confirming single template conditions in PCR1, and successful production of single chain peptide-MHC.

### Example 25: Release and Analysis of Identifier-Tagged Single Chain Multimeric Peptide-MHC from Hydrogels

This Example demonstrates release of folded, identifier-tagged single chain peptide-MHC (sc-pMHC) multimers from hydrogels. sc-pMHC were generated using the methods of examples 21, 22, 23, and 24. The sc-pMHC multimers were bound to the hydrogels via DNA identifiers. sc-pMHC bound to hydrogels via DNA can be released from the hydrogels by digestion with any suitable nuclease. In this example, DNA was digested by benzonase (a non-specific endonuclease) or FokI (a restriction enzyme) in Cutsmart Buffer (NEB), with a 20 hour incubation at 22° C. Protein released by digestion was tested by ELISA to determine yield and folding. Detection was done with a 1:1333 dilution of either H1RP-conjugated anti-B2M (Biolegend) or a conformationally sensitive anti-HLA antibody (Santa Cruz), with an HEK-produced sc-pMHC as a standard. ELISA confirmed release of highly folded sc-pMHC multimer (FIG. 18B). Protein released by digestion was also tested by Western Blot, with electrophoresis on a 3-8% Tris-Acetate gel, blotting to nitrocellulose, blocking with PBS plus 3% BSA, and detection with 1 μg/mL rat anti-Flag (Biolegend) primary and 1:1000 Alexa647 conjugated Anti-Rat IgG secondary (Invitrogen). The slow migration of FokI-released sc-pMHC multimer relative to benzonase-released sc-pMHC, or relative to supernatant from the in vitro transcription/translation supernatant, demonstrates successful tagging of the sc-pMHC with a nucleic acid identifier (FIG. 18C).

### Example 26: Functional Analysis of Single Chain Multimeric Peptide-MHC Produced in Hydrogels/Drops

This Example demonstrates sc-pMHC produced by methods of the disclosure bind specifically to cognate T cells. sc-pMHC released from hydrogels as described in example 25 were confirmed to bind specifically to cognate peptide-expanded T cells by flow cytometry and single cell encapsulation/sequencing.

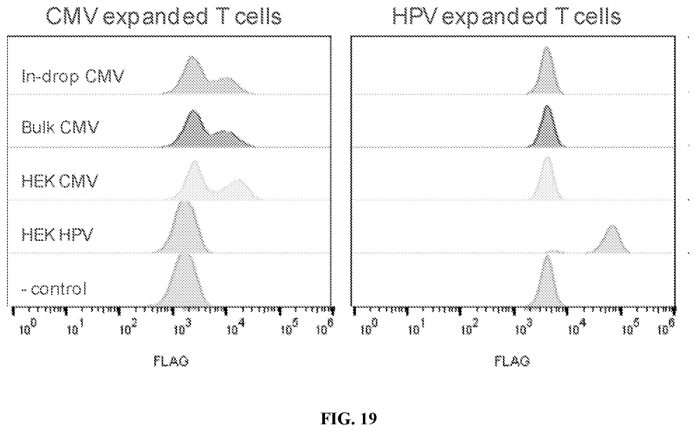

For flow, 10⁵ donor T cells that had been expanded with either HPV peptide or CMV peptide were stained with sc-pMHC multimers produced from a CMV peptide-encoding template either in bulk solution or in drops as described above. Control proteins corresponding to multimeric CMV or HPV pMHC produced in HEK cells were also used for staining. All pMHC were diluted in PBS plus 10% FBS, and anti-Flag-APC (Biolegend) was used as secondary. As shown in FIG. 19, CMV sc-pMHC multimers produced in hydrogels/drops displayed similar staining of CMV-expanded T cells as those produced in bulk or from HEK cells. Despite the fact that the HPV-expanded T cells were close to 100% positive for staining with HEK-produced HPV pMHC multimers, there was no apparent staining of the drop-produced CMV pMHC multimers on these cells, confirming specificity.

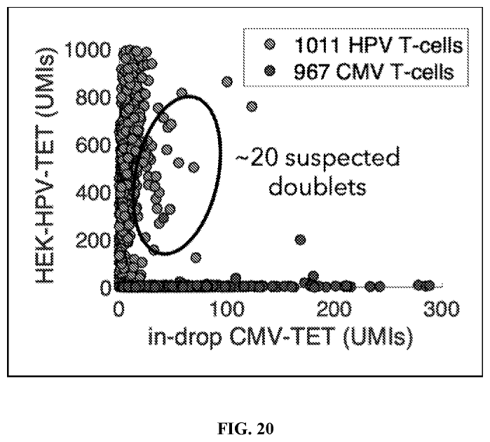

For single cell sequencing, CMV sc-pMHC multimers produced in drops with a self-identifying nucleic acid identifier tag as described in Examples 21-25 were treated with T7 exonuclease (NEB). The CMV sc-pMHC multimers were then mixed with HEK-produced HPV pMHC multimers, which were labeled with a distinct identifier. This antigen mixture was used to stain a mixture of HPV and CMV-expanded T cells, which were subsequently subjected to single cell sequencing. The single cell sequencing demonstrated excellent specificity of the hydrogel/drop-produced CMV sc-pMHC multimers. As shown in FIG. 20, UMIs corresponding to drop-produced CMV pMHC are associated with T cells that were expanded with CMV peptide, and not those expanded with HPV.

What is claimed is:

1. A peptide library comprising a plurality of peptides, wherein the plurality of peptides comprise more than 1,000, more than 2,000, more than 5,000, more than 10,000, more than 10⁶, more than 10⁷, more than 10⁸, more than 10⁹, or more than 10¹⁰ unique peptides.

2. The peptide library of claim 1, wherein the plurality of peptides comprises a plurality of antigens.

3. The peptide library of any one of claims 1-2, wherein the plurality of peptides comprises a plurality of pMHC multimers.

4. The peptide library of any one of claims 1-3, wherein the plurality of peptides comprises a plurality of sc-pMHCs.

5. The peptide library of any one of claims 1-4, wherein a peptide of the plurality is attached to a nucleotide sequence.

6. The peptide library of claim 5, wherein the nucleotide sequence is an identifier.

7. The peptide library of any one of claims 5-6, wherein the nucleotide sequence encodes the nucleotide sequence of the peptide.

8. The peptide library of any one of claims 5-7, wherein the nucleotide sequence is from 25 nucleotides to 500 nucleotides in length.

9. The peptide library of any one of claims 5-8, wherein the nucleotide sequence is from 80 nucleotides to 120 nucleotides in length.

10. The peptide library of any one of claims 3-9, wherein the MHC of the pMHC is MHC-I.

11. The peptide library of any one of claims 3-10, wherein the MHC of the pMHC is HLA-A, HLA-B, HLA-C, HLA-E, HLA-F, or HLA-G.

12. The peptide library of any one of claims 3-11, wherein the MHC of the pMHC is HLA-DPA1, HLA-DPB1, HLA-DQA1, HLA-DQB1, HLA-DRA, or HLA-DRB1.

13. A peptide subset library comprising the plurality of peptides of any one of claims 1-12, wherein proliferation of a T cell is induced upon binding of a peptide of the plurality of peptides of any one claims 1-12 to a TCR of the T cell.

14. A peptide subset library comprising the plurality of peptides of any one of claims 1-12, wherein cytotoxicity of a T cell is induced upon binding of a peptide of the plurality of peptides of any one claims 1-12 to a TCR of the T cell.

15. A peptide subset library comprising the plurality of peptides of any one of claims 1-12, wherein suppression of a T cell is induced upon binding of a peptide of the plurality of peptides of any one claims 1-12 to a TCR of the T cell.

16. A peptide subset library comprising the plurality of peptides of any one of claims 1-12, wherein suppression by a T cell is induced upon binding of a peptide of the plurality of peptides of any one claims 1-12 to a TCR of the T cell.

17. A peptide subset library comprising the plurality of peptides of any one of claims 1-12, wherein cytokine production of a T cell is induced upon binding of a peptide of the plurality of peptides of any one claims 1-12 to a TCR of the T cell.

18. A method of isolating lymphocyte-peptide pairs comprising:
(a) contacting a plurality of lymphocytes with a peptide library, wherein the peptide library has a diversity of more than 1000; and
(b) generating a plurality of compartments, wherein a compartment of the plurality comprises (i) a lymphocyte of the plurality of lymphocytes bound to a peptide of the peptide library, and (ii) a capture support.

19. The method of claim 18, wherein the lymphocyte is a T cell, B cell, or NK cell.

20. The method of any one of claims 18-19, wherein the peptide library has a diversity of more than 2,000, more than 5,000, more than 10,000, more than 10⁶, more than 10⁷, more than 10⁸, more than 10⁹, or more than 10¹⁰ unique peptides.

21. The method of any one of claims 18-20, wherein the plurality of peptides comprises a plurality of antigens.

22. The method of any one of claims 18-21, wherein the plurality of peptides comprises a plurality of pMHC multimers.

23. The method of any one of claims 18-22, wherein the plurality of peptides comprises a plurality of sc-pMHCs.

24. The method of any one of claims 18-23, wherein a peptide of the plurality is attached to a nucleotide sequence.

25. The method of claim 24, wherein the nucleotide sequence is an identifier.

26. The method of any one of claims 24-25, wherein the nucleotide sequence encodes the nucleotide sequence of the peptide.

27. The method of any one of claims 24-26, wherein the nucleotide sequence is from 25 nucleotides to 500 nucleotides in length.

28. The method of any one of claims 24-27, wherein the nucleotide sequence is from 80 nucleotides to 120 nucleotides in length.

29. The method of any one of claims 22-28, wherein the MHC of the pMHC is MHC-I.

30. The method of any one of claims 22-29, wherein the MHC of the pMHC is HLA-A, HLA-B, HLA-C, HLA-E, HLA-F, or HLA-G.

31. The method of any one of claims 22-30, wherein the MHC of the pMHC is HLA-DPA1, HLA-DPB1, HLA-DQA1, HLA-DQB1, HLA-DRA, or HLA-DRB1.

32. A method of identifying a lymphocyte-peptide pair comprising:
(a) contacting a plurality of lymphocytes with a library of peptides, wherein the library of peptides has a diversity of more than 1000;
(b) compartmentalizing a lymphocyte of the plurality of lymphocytes bound to a peptide of the library of peptides in a single compartment, wherein the peptide comprises a unique peptide identifier; and
(c) determining the unique peptide identifier for each peptide bound to the compartmentalized lymphocyte.

33. The method of claim 32, wherein the lymphocyte is a T cell, B cell, or NK cell.

34. The method of any one of claims 32-33, wherein the peptide library has a diversity of more than 2,000, more than 5,000, more than 10,000, more than 10⁶, more than 10⁷, more than 10⁸, more than 10⁹, or more than 10¹⁰ unique peptides.

35. The method of any one of claims 32-34, wherein the plurality of peptides comprises a plurality of antigens.

36. The method of any one of claims 32-35, wherein the plurality of peptides comprises a plurality of pMHC multimers.

37. The method of any one of claims 32-36, wherein the plurality of peptides comprises a plurality of sc-pMHCs.

38. The method of any one of claims 36-37, wherein the MHC of the pMHC is MHC-I.

39. The method of any one of claims 36-38, wherein the MHC of the pMHC is HLA-A, HLA-B, or HLA-C.

40. The method of any one of claims 36-39, wherein the MHC of the pMHC is HLA-DPA1, HLA-DPB1, HLA-DQA1, HLA-DQB1, HLA-DRA, or HLA-DRB1.

41. The method of any one of claims 32-40, wherein the lymphocyte-peptide pair is a TCR-antigen pair.

42. The method of any one of claims 32-41, further comprising determining an identity of a receptor on the compartmentalized lymphocyte.

43. The method of claim 42, wherein the determining the identity comprises sequencing a variable region, hypervariable region, or complementarity determining region (CDR) of a TCR, BCR, or antibody.

44. The method of claim 43, wherein the CDR is a CDR1, CDR2, or CDR3 of a TCR alpha chain, TCR beta chain, TCR gamma chain, TCR delta chain, antibody heavy chain, or antibody light chain.

45. A method of using an unbiased peptide library, the method comprising contacting a sample with the unbiased peptide library comprising a plurality of peptides, wherein the plurality of peptides comprise more than 100, more than 1,000, more than 2,000, more than 5,000, more than 10,000, more than 10⁶, more than 10⁷, more than 10⁸, more than 10⁹, or more than 10¹⁰ unique peptides.

46. The method of claim 45, wherein the unbiased peptide library is generated from a genome of an organism.

47. The method of claim 45, wherein the unbiased peptide library is generated from a transcriptome of an organism.

48. The method of claim 45, wherein the unbiased peptide library is generated from a proteome from an organism.

49. The method of claim 45, wherein the unbiased peptide library is generated from a peptide or protein from an organism.

50. The method of claim 45, wherein the unbiased peptide library is generated from an epitope from an organism.

51. The method of claim 45, wherein the unbiased peptide library is generated from differential sequences between genomes.

52. The method of claim 45, wherein the unbiased peptide library is generated from differential sequences between transcriptomes.

53. The method of claim 45, wherein the unbiased peptide library is generated from differential sequences between proteomes.

54. The method of any one of claims 51-53, wherein the differential sequences comprise sequences from a diseased cell versus a healthy cell.

55. The method of any one of claims 51-54, wherein the differential sequences comprise sequences from a cancerous cell versus a healthy cell.

56. The method of claim 45, wherein the unbiased peptide library is generated from homologous sequences between genomes.

57. The method of claim 45, wherein the unbiased peptide library is generated from homologous sequences between transcriptomes.

58. The method of claim 45, wherein the unbiased peptide library is generated from homologous sequences between proteomes.

59. The method of any one of claims 56-58, wherein the homologous sequences comprise sequences from a diseased cell versus a healthy cell.

60. The method of any one of claims 56-59, wherein the homologous sequences comprise sequences from a cell involved in autoimmunity versus a healthy cell.

61. The method of claim 45, wherein the unbiased peptide library comprises an unbiased pMHC multimer library.

62. The method of claim 45, wherein the unbiased peptide library comprises an unbiased sc-pMHC library.

63. The method of claim 61, wherein antigens of the pMHC multimers of the unbiased pMHC multimer library are unbiased.

64. The method of claim 62, wherein antigens of the sc-pMHC of the unbiased sc-pMHC library are unbiased.

65. A composition comprising pMHC multimer attached to a unique identifier.

66. The composition of claim 65, wherein the pMHC multimer is a sc-pMHC.

67. The composition of claim 65 or claim 66, wherein the unique identifier is a nucleic acid.

68. The composition of any one of claims 65-67, wherein the unique identifier is a self-identifier.

69. The composition of any one of claims 65-67, wherein the unique identifier is not a self-identifier.

70. The composition of any one of claims 65-69, wherein the unique identifier is from 25 nucleotides to 120 nucleotides in length.

71. A compartment comprising:
(a) a sequence encoding a sc-pMHC; and
(b) a T cell.

72. A composition comprising:
(a) a hydrogel bead; and
(b) a nucleic acid attached to the hydrogel bead, wherein the nucleic acid encodes a peptide.

73. The composition of claim 72, further comprising a second nucleic acid attached to the hydrogel bead, wherein the second nucleic acid comprises an identifier.

74. The composition of claim 73, wherein the identifier is a self-identifier.

75. The composition of claim 73, wherein the identifier is not a self-identifier.

76. The composition of any one of claims 72-75, further comprising the peptide, wherein the peptide is attached to the hydrogel bead.

77. The composition of any one of claims 73-75, further comprising the peptide, wherein the peptide is attached to a third nucleic acid and the third nucleic acid attached to the hydrogel bead.

78. The composition of claim 77, wherein the third nucleic acid comprises the identifier.

79. The composition of any one of claims 72-78, wherein the hydrogel bead is encapsulated in a droplet.

80. The composition of any one of claims 72-79, wherein the peptide comprises a sc-pMHC.

81. The composition of claim 80, wherein the MHC of the sc-pMHC is MHC-I.

82. The composition of claim 80, wherein the MHC of the sc-pMHC is HLA-A, HLA-B, HLA-C, HLA-E, HLA-F, or HLA-G.

83. The method of claim 80, wherein the MHC of the sc-pMHC is HLA-DPA1, HLA-DPB1, HLA-DQA1, HLA-DQB1, HLA-DRA, or HLA-DRB1.

84. A method of generating an identifier-tagged peptide, comprising:
(a) providing a capture support, wherein the capture support comprises an attached nucleic acid that comprises an identifier;
(b) attaching a peptide to the nucleic acid;
(c) separating the nucleic acid or a part thereof from the capture support, thereby releasing the identifier-tagged peptide.

85. The method of claim 84, wherein the identifier is a self-identifier.

86. The method of claim 84, wherein the identifier is not a self-identifier.

87. The method of any one of claims 84-86, wherein the peptide comprises an antigen.

88. The method of any one of claims 84-87, wherein the peptide comprises a sc-pMHC.

89. The method of claim 88, wherein the MHC of the sc-pMHC is MHC-I.

90. The method of claim 88, wherein the MHC of the sc-pMHC is HLA-A, HLA-B, HLA-C, HLA-E, HLA-F, or HLA-G.

91. The method of claim 88, wherein the MHC of the sc-pMHC is HLA-DPA1, HLA-DPB1, HLA-DQA1, HLA-DQB1, HLA-DRA, or HLA-DRB1.

92. The method of any one of claims 84-91, wherein the separating comprises enzymatic digestion.

93. The method of any one of claims 84-92, wherein the capture support is encapsulated in a droplet.

94. The method of any one of claims 84-93, wherein the nucleic acid is from 25 nucleotides to 500 nucleotides in length.

95. The method of any one of claims 84-93, wherein the nucleic acid is from 80 nucleotides to 120 nucleotides in length.

96. A method of generating a peptide library, comprising generating a plurality of identifier-tagged peptides via the method of any one of claims 84-95.

97. A method of generating an identifier-tagged peptide, comprising:
(a) providing a capture support, wherein the capture support has a first nucleic acid attached that encodes a peptide and a second nucleic acid attached that comprises an identifier;
(b) producing the peptide;
(c) attaching the peptide to the second nucleic acid, thereby generating the identifier-tagged peptide; and
(d) separating the second nucleic acid or a part thereof from the capture support, thereby releasing the identifier-tagged peptide.

98. The method of claim 97, wherein the identifier is a self-identifier.

99. The method of claim 97, wherein the identifier is not a self-identifier.

100. The method of any one of claims 97-99, wherein the producing comprises in vitro transcription and translation.

101. The method of any one of claims 97-100, wherein the peptide comprises an antigen.

102. The method of any one of claims 97-101, wherein the peptide comprises a sc-pMHC.

103. The method of claim 102, wherein the MHC of the sc-pMHC is MHC-I.

104. The method of claim 102, wherein the MHC of the sc-pMHC is HLA-A, HLA-B, HLA-C, HLA-E, HLA-F, or HLA-G.

105. The method of claim 102, wherein the MHC of the sc-pMHC is HLA-DPA1, HLA-DPB1, HLA-DQA1, HLA-DQB1, HLA-DRA, or HLA-DRB1.

106. The method of any one of claims 97-105, wherein the separating comprises enzymatic digestion.

107. The method of any one of claims 97-106, wherein the capture support is encapsulated in a droplet.

108. The method of any one of claims 97-107, wherein the second nucleic acid is from 25 nucleotides to 500 nucleotides in length.

109. The method of any one of claims 97-107, wherein the second nucleic acid is from 80 nucleotides to 120 nucleotides in length.

110. A method of generating a peptide library, comprising generating a plurality of identifier-tagged peptides via the method of any one of claims 97-109.
